(19) United States
(12) Patent Application Publication
(10) Pub. No.: US 2022/0365074 A1
(43) Pub. Date: Nov. 17, 2022

(54) METHODS AND SYSTEMS FOR DETERMINING VIRUSES SUCH AS CORONAVIRUSES USING pH

(71) Applicant: Analiza, Inc., Bay Village, OH (US)

(72) Inventors: Alan Bauer, Jerusalem (IL); Arnon Chait, Bay Village, OH (US)

(73) Assignee: Analiza, Inc., Bay Village, OH (US)

(21) Appl. No.: 17/212,080

(22) Filed: Mar. 25, 2021

Related U.S. Application Data
(60) Provisional application No. 63/000,435, filed on Mar. 26, 2020.

(51) Int. Cl. G01N 33/50; G01N 33/52; G01N 15/14

(52) U.S. Cl. CPC G01N 33/5091; G01N 33/52; G01N 2015/1006; G01N 2333/165; G01N 15/1434

(57) ABSTRACT

Certain aspects of the present disclosure generally relates to devices and methods for determining, treating, and/or isolating cells of interest, e.g., within a mixture of cells. In some cases, the cells may be cells infected with viruses, such as coronaviruses. In some embodiments, blood samples (or other biological fluids, such as saliva) may be treated with a pH-sensitive entity. The pH-sensitive entity may be one that is able to change color or otherwise produce a signal in suitable internal environments. For example, cells infected by viruses, such as coronaviruses, may have differences in intracellular pH compared to other cells, which can be detected, for example, using pH-sensitive entities. In certain embodiments, the cells may be sorted based on such signaling entities; for example, illumination of cells in a suitable machine for sorting cells (e.g., using fluorescent light) may allow determination of the cells, which may also be recovered or isolated for further manipulation in some cases.

### RELATED APPLICATIONS

This application claims the benefit of U.S. Provisional Patent Application Ser. No. 63/000,435, filed Mar. 26, 2020, entitled “Methods and Systems for Determining Viruses such as Coronaviruses Using pH,” by Bauer, et al., which is incorporated herein by reference in its entirety.

### FIELD

Certain aspects of the present disclosure generally relates to devices and methods for determining, treating, and/or isolating cells of interest, e.g., within a mixture of cells. In some cases, the cells may be cells infected with viruses, such as coronaviruses.

### BACKGROUND

Coronaviruses are a group of viruses that cause diseases in mammals and birds. In humans, coronaviruses cause respiratory tract infections that are typically mild, such as the common cold, though rarer forms such as SARS, MERS and COVID-19 can be lethal. Coronaviruses are enveloped viruses with a positive-sense single-stranded RNA genome and a nucleocapsid of helical symmetry. The genome size of coronaviruses ranges from approximately 27 to 34 kilobases. The name coronavirus is derived from the Latin corona, meaning “crown” or “halo,” which refers to the characteristic appearance of the virus particles: they have a fringe reminiscent of a crown or of a solar corona.

### SUMMARY

Certain aspects of the present disclosure generally relates to devices and methods for determining, treating, and/or isolating cells of interest, e.g., within a mixture of cells. In some cases, the cells may be cells infected with viruses, such as coronaviruses. The subject matter of the present disclosure involves, in some cases, interrelated products, alternative solutions to a particular problem, and/or a plurality of different uses of one or more systems and/or articles.

One aspect is generally directed to a method of determining a coronavirus infection. In one set of embodiments, the method comprises exposing a fluid containing cells arising from a subject to a pH-sensitive entity, wherein the pH-sensitive entity has a first state within cells infected by the coronavirus and a second state within cells not infected by the coronavirus; and determining the pH-sensitive entity internally within at least some of the cells within the fluid.

Another aspect is generally directed to a device. In one set of embodiments, the device comprises a fluid comprising cells arising from a subject; a pH-sensitive entity internalized within the cells, wherein the pH-sensitive entity has a first state within cells infected by the coronavirus and a second state within cells not infected by the coronavirus; and a cell cytometer containing the fluid.

Yet another aspect is generally directed to a method of determining a coronavirus infection, comprising: exposing a fluid containing cells arising from a subject to a pH-sensitive entity, wherein the pH-sensitive entity has a first state within cells infected by the coronavirus and a second state within cells not infected by the coronavirus; and determining the pH-sensitive entity internally within at least some of the cells within the fluid.

Still another aspect is generally directed to a device, comprising a fluid comprising cells arising from a subject; a pH-sensitive entity internalized within the cells, wherein the pH-sensitive entity has a first state within cells infected by the coronavirus and a second state within cells not infected by the coronavirus; and a cell cytometer containing the fluid.

In another aspect, the present disclosure encompasses methods of making one or more of the embodiments described herein. In still another aspect, the present disclosure encompasses methods of using one or more of the embodiments described herein.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 shows a method according to one embodiment of the disclosure;

FIG. 2 shows a method according to another embodiment of the disclosure;

FIGS. 3A-3D shows a method according to yet another embodiment of the disclosure; and

FIG. 4 shows a flowchart for a method according to one embodiment of the disclosure.

### DETAILED DESCRIPTION

Certain aspects of the present disclosure generally relates to devices and methods for determining, treating, and/or isolating cells of interest, e.g., within a mixture of cells. In some cases, the cells may be cells infected with viruses, such as coronaviruses. In some embodiments, blood samples (or other biological fluids, such as saliva) may be treated with a pH-sensitive entity. The pH-sensitive entity may be one that is able to change color or otherwise produce a signal in suitable internal environments. For example, cells infected by viruses, such as coronaviruses, may have differences in intracellular pH compared to other cells, which can be detected, for example, using pH-sensitive entities. In certain embodiments, the cells may be sorted based on such signaling entities; for example, illumination of cells in a suitable machine for sorting cells (e.g., using fluorescent light) may allow determination of the cells, which may also be recovered or isolated for further manipulation in some cases.

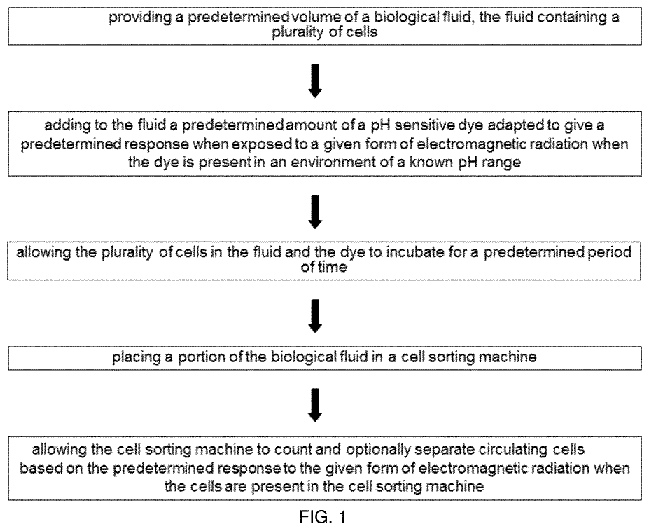

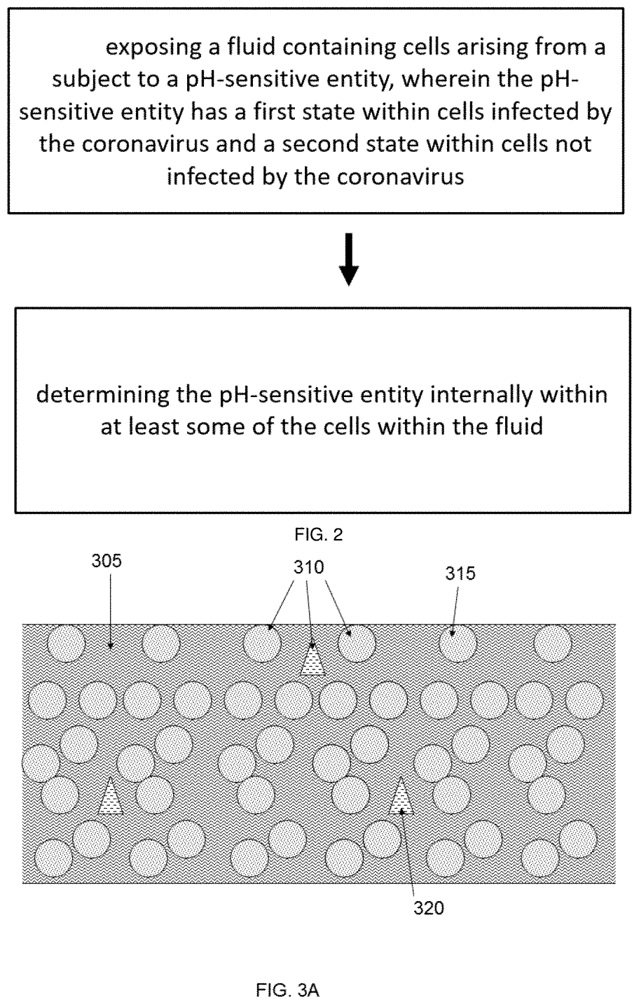

For example, the present disclosure, in some embodiments thereof, relates to systems and devices for determining cells that are infected with viruses, such as coronaviruses. The present disclosure, in some embodiments, also provides for isolation of such cells, e.g., for potential genetic or other investigation. Certain embodiments of the present disclosure could be employed for a wide variety of applications including, but not limited to, testing to determine infection, e.g., by a virus such as a coronavirus, individualized medicine to include customization of therapy, genetic testing to assess potential medical conditions, or other purposes. Examples of such embodiments include, but are not limited to, those shown in FIGS. 1 and 2.

Some embodiments of the disclosure are generally directed to the determination of cells, within a larger number or population of cells, for instance, if not all of the cells within the fluid are infected. In some cases, less than about 1% of the population of cells may be the cells which are desired to be determined and/or isolated; in other cases, this may be less than about 0.1%, less than about 0.01%, less than about 0.001%, less than about 10⁻⁴%, less than about 10⁻⁵%, or less than about 10⁻⁶%. The cells may be suspended in blood, or another fluid (e.g., saline, cell media, amniotic fluid, etc.).

Certain aspects of the present disclosure is generally directed to systems and methods for determining viruses, such as coronaviruses. A sample, e.g., of a biological fluid taken from a subject, may be analyzed to determine whether a species of virus is present (e.g., SARS, MERS, COVID-19, etc.), or a type of virus is present (e.g., a coronavirus). In addition in some cases, different types of viruses may be distinguished (e.g., a coronavirus versus an influenza virus.

The sample of biological fluid may include fluids such as whole blood, blood serum, blood plasma, saliva, sputum, urine, CNS fluid, breast nipple aspirate fluid, cerebral spinal fluid, semen, or the like. The subject that the biological fluid is taken from may be human, or non-human, e.g., a non-human mammal. Non-human mammals include, but are not limited to, a dog, cat, horse, cow, pig, sheep, goat, chicken, primate, rat, and mouse. In some cases, the subject is one that is suspected of being infected with a virus. Other example of fluids and subjects are described herein.

A variety of different viruses may be determined, in accordance with various embodiments. Non-limiting examples of viruses, including infectious viruses, include coronaviruses or influenza viruses. Other non-limiting examples include adenoviruses, coxsackieviruses, Epstein-Barr viruses, hepatitis viruses (A, B, and C), herpes simplex viruses (types 1 and 2), cytomegaloviruses, herpes viruses (type 8), HIV, measles viruses, mumps viruses, papilloma viruses, parainfluenza viruses, polioviruses, rabies viruses, respiratory syncytial viruses, rubella viruses, varicella-zoster viruses, etc.

In some embodiments, a coronavirus may be determined. Examples of coronaviruses include, but are not limited to, HCoV-229E, HCoV-OC43, SARS-CoV, HCoV-NL63, HKU1, MERS-CoV, or SARS-CoV-2. In some cases, one or more proteins of the coronavirus may be used to determine the virus, e.g., by interaction with a virus-binding moiety or a targeting species, such as are discussed herein. Examples of such proteins include peplomers, envelope proteins, membrane proteins, nucleocapsids, spike glycoproteins, hemagglutinin-esterase dimers (HE), or the like. In addition in some cases, the nuclear material of the virus (for example, RNA) may be determined, e.g., by interaction with a virus-binding moiety or a targeting species.

In some embodiments, an influenza virus may be determined. Influenza viruses include genera such as Influenza virus A, Influenza virus B, Influenza virus C, Influenza virus D, Isavirus, Thogotovirus, and Quaranjavirus. Examples of influenza A viruses include H1N1, H1N2, H2N2, H3N1, H3N2, H3N8, H5N1, H5N2, H5N3, H5N8, H5N9, H7N1, H7N2, H7N3, H7N4, H7N7, H7N9, H9N2, H10N7, etc. Examples of Influenza B virus include Victoria and Yamagata. In some cases, one or more proteins of the influenza virus may be used to determine the virus, e.g., by interaction with a virus-binding moiety or a targeting species, such as are discussed herein. Examples of such proteins include hemagglutinin, neuraminidase, membrane proteins, glycoproteins, nucleocapsids, etc. In addition in some cases, the nuclear material of the virus (for example, RNA) may be determined, e.g., by interaction with a virus-binding moiety or a targeting species.

In some cases, the present disclosure generally relates to devices and methods for determining, treating, and/or isolating cells, e.g., within a mixture of cells. In some embodiments, the present disclosure is generally directed to devices and methods for allowing an uncharged molecule to enter a cell and be converted into a charged molecule, for instance, in cells of interest. The charged molecule, unlike its uncharged precursor, may be one that cannot pass back out through the cellular membrane, and thus may remain “trapped” within the cell. The charged molecule may be determinable, for example, via fluorescence, and/or the charged molecule may include therapeutic features such as radioactive ionization and/or enzyme inhibitory behavior. In some aspects, properties of target cells, such as high pH and/or unique enzymatic features, may allow for the specific conversion of uncharged molecule to charged molecules in cells of interest. For example, the cells may be cells infected with viruses, such as coronaviruses, where the cells infected by viruses may have differences in intracellular pH compared to other cells, which can be determined.

For example, certain aspects of the present disclosure are generally directed to systems and methods for introducing an uncharged compound into a cell, and allowing the uncharged compound to become charged while in the cell. In some cases, the compound, once charged, is unable to readily exit the cell, and thus may remain lodged within the cell. The compound may be detectable (e.g., fluorescent or radioactive), and/or in some cases, the compound may have desired effects on the cell (e.g., by treating the cell or killing the cell). In some cases, if a population of cells is present, some cells may be able to convert the uncharged compound into a charged compound, while other are unable (or less able) to do so. In this way, different types of cells may be distinguished, e.g., cells infected by a virus and cells not infected by the virus.

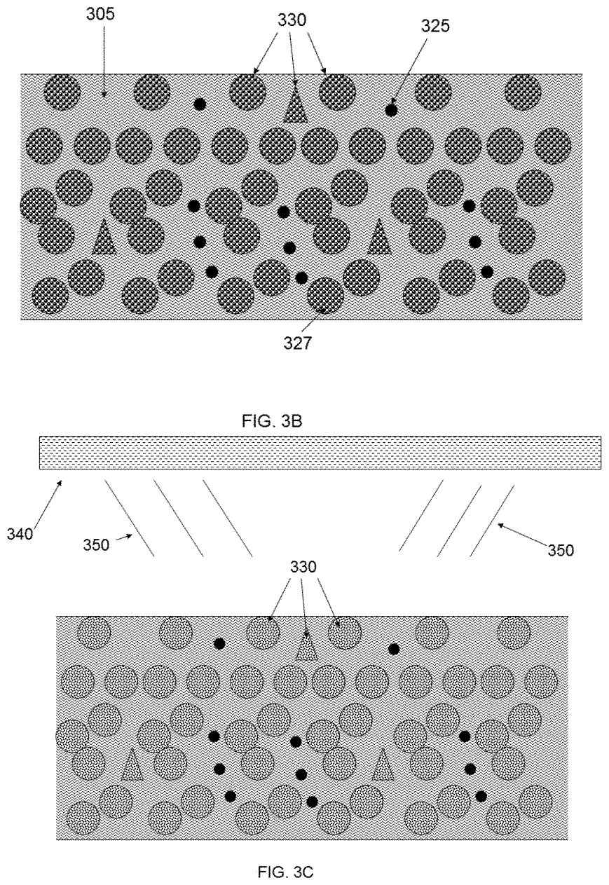

Non-limiting examples of methods according to various embodiments of the disclosure are shown in FIGS. 1 and 2. As another non-limiting example, FIGS. 3A-3D show schematic views of aspects of a device according to one set of embodiments. In this example, fluid sample 305 includes cells 310, where a plurality of said cells 310 are non-target cells 315 (e.g., uninfected cells) and a small percentage are target cells 320 (e.g., cells infected with a virus, such as a coronavirus).

In FIG. 3B, pH-sensitive entity 325 is added to the fluid sample 305 and allowed to incubate with the fluid sample 305 for a predetermined period of time, e.g., between 1 minute and two hours. As shown in FIG. 3B, a portion of entity 325 enters cells due to the entity's ability to pass through cellular membranes. The cell-internalized pH-sensitive entity is shown as small black balls 327 in cells 330 containing the entity.

FIG. 3C shows entity-containing cells 330 in cell sorting machine 340 adapted to provide electromagnetic radiation 350 in the form of fluorescent light, in this particular example. FIG. 3D shows a view of the entity-containing cells 330 as seen after illumination with fluorescent light. Cells 360 show a predetermined color whereas the other cells 370 either show no color or show non-target color, for instance, based on the response of the entity to their intercellular pH levels. As a non-limiting example, cells 360 may fluoresce at a known wavelength when treated with a light of another, prescribed wavelength, whereas white blood cells and other cells found in the fluid sample 305 show other colors or no colors at all. Other examples are discussed herein. Determination of cells 360 allow for detection, identification, counting, sorting, and/or other manipulation of the cells, e.g., using suitable cell cytometers or the like. Subsequent analysis of the cells may include, but is not limited to, genetic analysis, morphology analysis, cytopathology analysis, or biochemical treatment.

In one set of embodiments, as discussed, a signaling molecule, or other signaling entity, is added to the fluid containing the cells or the cells are otherwise exposed to the signaling molecule or entity in some fashion. The signaling entity may be internalized by the cells, e.g., actively or passively. In some cases, once internalized, the signaling entity may change in some fashion in some of the cells (e.g., the cells of interest), while the signaling molecule may not change (or may change in a different way) in other cells. As a non-limiting example, the signaling entity may be pH-sensitive and/or may produce different “colors” or emissions at different pHs. The cells may then be determined and/or isolated using any suitable technique known in the art, based on the signaling entity, as discussed herein. For instance, in one set of embodiments, the cells may be separated using a flow cytometer or a cell sorter machine, such as a fluorescence-activated cell sorting (FACS) system. Thus, for example, cells having one intracellular pH (representing a first cell type, such as a tumor cell or a fetal cell) may be separated from cells having a different intracellular pH (representing cells of a second type, such as non-tumor or maternal cells).

Without wishing to be bound by any theory, it should be understood that certain cell types, such as cells infected by a virus, may engage in metabolic behavior that significantly alters their internal cellular pH, e.g., to distinguish these cells from the majority of cells in a that are not infected, and/or there may be other changes that modify the polarity of their cytoplasm that can be determined or observed as changes in pH. It is believed that this is reflective of reactions and processes going on in such cells, and may thus provide information not reflected in other aspects of these cells, such as their relative size or relative abundance of certain cell membrane antigens. For example, in the case of cells infected with a virus, a change in intracellular pH may indicate the existence of specific functional attributes that may be useful for clinical applications, e.g., due to the metabolic processes occurring as the virus causes the cell to produce and assemble components of the virus for viral reproduction. For instance, in some cases, the intracellular pH of normal cells may be between about 6.8 and about 6.9, while the pH of the infected cells may be lower or higher than this. In some cases, these pH changes may be significant, e.g., resulting in a change of at least about 0.1, at least about 0.2, at least about 0.3, at least about 0.4, at least about 0.5, at least about 0.7, at least about 1, at least about 1.2, at about 1.5, at least about 1.7, at least about 2, or more pH units, relative to normal intracellular pHs. In some cases, the pH changes may be significant. For example, there may be a change of at least about 0.5, at least about 1, at about 1.5, at least about 2, or more pH units, relative to normal intracellular pHs. In one set of embodiments, the me

In addition, it should also be understood that fluids other than blood may be analyzed in other embodiments of the disclosure; for example, the cells may be present in other fluids such as blood, saliva, blood serum, vaginal fluid, cervical fluid, cerebral spinal fluid, urine, nipple aspirate, phlegm, sputum, pleural abdominal exudate or transudate, amniotic fluid, saline, cell media, water, or the like. In some cases, the fluid may be one that arises biologically, e.g., from an organism such as a human. The cells may be human and/or non-human cells. For example, in one embodiment, non-human cells present within blood (or other fluid) may be separated from human cells, e.g., on the basis of intracellular pH or other conditions.

If a signaling entity is present, the signaling entity may generally be a material that responds to electromagnetic radiation or other energy directed at it. In some cases, the signaling entity can specifically bind to an analyte; however, in other cases, the signaling entity can bind nonspecifically or otherwise interact with various analytes, or to other species (e.g., H⁺ in the case of some pH-sensitive entities). The signaling entity may be a single type of molecule, or a plurality of different types of molecules in some cases. Color generation or fluorescence is one of a number of possible responses including, but not limited to, energy release, or chemical reactivity. Thus, a signaling entity, as used herein, is not limited to only color changes. It should also be understood that color generally refers to any response of the entity to treatment with electromagnetic radiation. Fluorescence, light, Raman, or other quantum-related phenomena are non-limiting examples of a response that may be referred to as a “color” change. Examples of suitable electromagnetic radiation include, but are not limited to, white light, laser light at a predetermined at least one wavelength, visible light at at least one wavelength, fluorescent light, X-ray radiation, microwave radiation, etc. or a combination of different forms of electromagnetic radiation, including but not limited to any combination of any of these. Those of ordinary skill in the art will be able to readily determine suitable electromagnetic radiation based on the signaling entity used.

In addition, it should be understood that in some cases, the fluid (e.g., blood) may undergo pre-treatment with chemicals, physical conditions, etc., for example, prior to or simultaneously with the addition of one or more signaling entities. For example, the fluid may be filtered, treated with an anticoagulant (e.g., citrate or heparin), acidified, centrifuged, or the like. As another example, the fluid may be exposed to one or more buffers. In some cases, the buffers may include buffers at different pH's.

In one set of embodiments, the signaling entity is pH-sensitive. pH sensitivity, as discussed herein, includes not only the usual definition of hydrogen ion activity in solution, but also a more extended description that includes solution polarity and the like, at least in some embodiments. The pH-sensitive entity may have at least a first color (or other determinable state) at a first pH and a second color (or other determinable state) at a second pH different from the first pH. The first pH and the second pH may be separated by at least about 0.5, at least about 1, at about 1.5, at least about 2, at least about 2.5, at least about 3, at least about 4, or at least about 5 pH units. In some cases, certain cells, such as some types of cancer cells or fetal cells, may exhibit differences in intracellular pH compared to other cells, which can be detected using pH-sensitive signaling entities.

In some cases, the signaling entity may be able to permeate cellular membranes or otherwise enter a cell, e.g., into the cytoplasm. In some cases, the signaling entity can diffuse passively across a cellular membrane; in other cases, however, the signaling entity enters a cell through active processes (e.g., via phagocytosis, pinocytosis, stimulation of cell-surface receptors, or the like). The signaling entity may also be able to adapt forms and color schemes that are reflective of the pH or solvent polarity environments associated with the inner regions of such cells, and in some cases, in response to modifying molecules that may be present in such regions (e.g., enzymes). The signaling entity may be biocompatible in some fashion, although in certain cases, the signaling entity need not be biocompatible; for example, exposure of the cells to the signaling entity may injure or kill the cells, although determination of the signaling entity may still occur.

The signaling entity may be excited in some fashion, e.g., using suitable electromagnetic energy, to allow for the identification or determination of such cells, e.g., due to the unique pH-associated color found in those cells. For example, the signaling entity may exhibit fluorescence, phosphorescence, a change in absorption (e.g., at particular wavelengths), or the like. Examples of suitable electromagnetic energy include, but are not limited to, white light, laser light at a predetermined at least one wavelength, visible light at at least one wavelength, fluorescent light, X-ray radiation, microwave radiation, etc. or a combination of any of these and/or other types of electromagnetic energy.

The following are non-limiting examples of pH-sensitive entities that may be used in various embodiments: 2′,7′-bis(2-carboxyethyl)-5(6)-carboxyfluorescein tetrakis(acetoxymethyl) ester (BCECF AM), 5(6)-carboxy-2′,7′-dichlorofluorescein, 5(6)-carboxyfluorescein, 5(6)-carboxyfluorescein diacetate, 5(6)-carboxyfluorescein N-hydroxysuccinimide ester, 3,6-diacetoxyphthalonitrile, 6,8-dihydroxy-1,3-pyrenedisulfonic acid disodium salt, Eosin diacetate, or naphthofluorescein. Other pH-sensitive entities may also be used in some cases. In some embodiments, the pH-sensitive entity may be one whose color profile at various pH value changes, and which has some ability to enter a cell, e.g., passively or through diffusion. In some cases, the pH-sensitive entity may be one that fluoresces after undergoing chemical modification via intracellular enzymes. Many such pH-sensitive entities can be obtained commercially. However, it should be understood that the pH-sensitive entities can also include not only pH dyes per se, but also other entities that show color changes or other discriminatory behavior relative to pH or the like.

In one set of embodiments, a signaling entity (e.g., one that is pH-sensitive) may be used to distinguish a target cell of interest (e.g., a cell infected with a virus, such as a coronavirus) from other surrounding cells that are not of interest (e.g., a cell not infected with a virus, etc.). Other examples are discussed herein. In some cases, for example, the signaling entity may have a first state in a first type of cell (e.g., a target cell, such as a cell infected with a virus) and a second state in a second type of cell (e.g., a cell not infected with a virus), where the first state and the second state are different; for instance, the first state may be fluorescent, while the second may be less fluorescent (or substantially less fluorescent).

As an example, with respect to a pH-sensitive entity, the first type of cell may have a first intracellular pH, and the second type of cell may have a second intracellular pH, where the pH's are different by at least about 0.1, at least about 0.2, at least about 0.3, at least about 0.4, at least about 0.5,at least about 0.7, at least about 1.0, at least about 1.2, at least about 1.5, at least about 1.7, at least about 2.0 pH units, or more in some cases. If the pH-sensitive entity has different states in the first and second cell types, then the cell types may be distinguished from each other, e.g., as is discussed herein.

For instance, at a first pH (e.g., the intracellular pH of a target cell), the pH-sensitive entity may be fluorescent, and at a second pH (e.g., the intracellular pH of a non-target cell), the pH-sensitive entity may be substantially less fluorescent, e.g., producing emissions at an intensity that is less than about 50%, less than about 20%, less than about 10%, less than about 5%, less than about 2%, or less than about 1% of the first pH. In some cases, the first pH and the second pH may differ by at least about 0.5 pH units, at least about 1.0 pH unit, at least about 1.5 pH units, at least about 2.0 pH units, or more in some cases. For example, the pH-sensitive entity may be fluorescent at a pH of greater than about 7.5 and substantially less fluorescent at a pH of less than about 7, the pH-sensitive entity may be fluorescent at a pH of greater than about 7 and substantially less fluorescent at a pH of less than about 6.5, the pH-sensitive entity may be fluorescent at a pH of greater than about 6.5 and substantially less fluorescent at a pH of less than about 6, the pH-sensitive entity may be fluorescent at a pH of greater than about 6 and substantially less fluorescent at a pH of less than about 5.5, etc. (In addition, in other embodiments, any of the roles discussed here may be reversed, e.g., the pH-sensitive entity may be fluorescent at a pH of a non-target cell and substantially less fluorescent at the pH of a target cell.)

By determining fluorescence of cells within a population of cells within a sample, the first and second cell types may be distinguishable from each other. In contrast, although pH-sensitive entities or other signaling entities have previously been used to study cells, such entities have not been used to distinguish different cell types from each other.

As mentioned, various embodiments of the present disclosure are generally directed to the determination and/or isolation of cells of interest from a population of cells. The volume of fluid containing the cells to be analyzed may be any suitable volume, for example, femtoliters, microliters, milliliters, liters, etc. In some cases, these cells may represent a very small part of the population of cells, as previously discussed. In certain embodiments, the cells may be determined, i.e., a population of cells is studied to identify whether certain cells are present, and/or how many of those cells are present. Thus, the determination may be qualitative and/or quantitative, in various applications. In certain embodiments, the cells of interest may be isolated from the population of cells. For example, these cells may be separated from the population of cells and placed at a first location (e.g., a collection chamber), while the other cells are placed at a second location (e.g., a second collection chamber), or perhaps discarded. In some cases, the isolated cells may be further analyzed, e.g., genetically, morphologically, cytopathologically, phenotypically, etc., e.g., as discussed herein. As a non-limiting example, the genetic analysis may include a search for genetic abnormalities such as chromosome defects.

According to one set of embodiments, the cells of interest may be ones that exhibit a change in intracellular pH or other internal characteristic. For example, infected cells often exhibit varying pH's, as compared to non-infected cells. Without wishing to be bound by any theory, it is believed that this may be due to altered metabolic states present within the infected cells, increased metabolism of the infected cells relative to non-infected cells, and/or other factors. Such cells may have lower intracellular pH at an alkaline level (e.g., around 7.4), compared to normal cells.

In some cases, as mentioned, changes in pH may occur due to the metabolic processes occurring as the virus causes the cell to produce and assemble components of the virus for viral reproduction. By determining abnormal pH's in cells, as compared to normal cells, cells infected with a virus, such as a coronavirus, may be determined in certain embodiments of the invention.

In addition, in one set of embodiments, the fluid may be acidified prior to (or after) exposure of the fluid to the pH-sensitive entity (or other signaling entity), e.g., to increase the acidity of the fluid (decrease the pH of the fluid). In one set of embodiments, the fluid may be acidified by exposure to a suitable acid, such as ethylenediaminetetraacetate acid, citric acid, ascorbic acid, dehydroascorbic acid, or the like. In some cases, the acids are those that are useful for preservation or to prevent blood coagulation, etc. The acid may be added in an amount able to decrease the pH of the fluid by at least about 0.2, at least about 0.3, at least about 0.4, at least about 0.5, at least about 0.7, at least about 1 pH units, or more in some cases. In some cases, the pH is not modified by more than about 3.0, about 2.5, about 2.0, about 1.5, or about 1.0 pH units. The acid may also be present in concentrations or pH's that are insufficient to cause extended or substantial cell death within the fluid.

In certain embodiments, the fluid may be acidified by waiting a sufficient time (e.g., with or without exposure to an acid); for example, the fluid may be kept in an open or closed container for at least about 3 hours, at least about 6 hours, at least about 9 hours, at least about 12 hours, at least about 18 hours, at least about 24 hours, at least about 30 hours, at least about 2 days, at least about 3 days, at least about 4 days, etc., and/or any range between any of these numbers. As a non-limiting example, a fluid such as blood may be kept in a container for between 24-30 hours. The fluid may be kept under such conditions at any suitable temperature, e.g., around body temperature (about 37° C.), around room temperature (about 25° C.), within a refrigerator (about 4° C.), etc. Without wishing to be bound by any theory, it is believed that cells within the fluid may continue to be metabolically active (e.g., producing acid by-products, such as lactic acid), or consume oxygen and/or produce carbon dioxide (which may form carbonic acid within the fluid), which may facilitate acidification of the fluid. Thus, in certain embodiments, the fluid may be acidified by waiting for a sufficient time.

With respect to fluid acidification, without wishing to be bound by any theory, it is believed that, somewhat counter-intuitively, acidification of the fluid may promote the ability to determine or distinguish target cells from other non-target cells within the fluid using a pH-sensitive entity, at least in certain embodiments of the disclosure. It is believed that certain cell types, such as cells infected with viruses, are more metabolically active and/or have the ability to resist acidification to a greater degree than other types of cells. Accordingly, by acidifying the fluid, cells of interest that can resist acidification can be more readily determined or distinguished, compared to other cells that cannot resist acidification to the same degree. Thus, even though a pH-sensitive entity is used in certain embodiments, the fluid may also be acidified, before and/or after exposure of the fluid to the pH-sensitive entity.

Once exposed to a suitable signaling entity, such as a pH-sensitive entity, cells exhibiting a first characteristic (such as a first pH, e.g., an intracellular pH) may be determined and/or isolated from cells exhibiting a second characteristic (such as a second pH, e.g., an intracellular pH). Many techniques for separating cells on the basis of a signaling entity are available. For example, in some embodiments, a cell sorting machine may be used. Cell sorting machines may include flow cytometry devices and the like. A cell cytometer or a flow cytometer generally describes a machine capable of identifying, determining, and/or counting cells based on a signal emitted from the cells. Cell cytometers, amongst other devices, allow rapid determination or counting of cells based on a differential response to, e.g., electromagnetic radiation. The count of cells may be reported using any suitable technique, for example, as absolute numbers (e.g., number of cells), as a density (e.g., count in a given volume of biological fluid), in proportion to other cells in the same fluid, or the like. This may suitable in some applications, e.g., for clinical purposes, assessing metastatic potential of tumor cells, or the like.

Cell cytometers may also include additional component to further sort cells based on such response, in certain instances. For example, the cell cytometer may incorporate multiple detection stages to provide negative or positive selection of cells. Computing devices and other elements may also be used for control of processes, as well as for data analysis and storage, in some cell sorting machines. Examples include a mobile computing device, smartphone, cellular phone, tablet computer, laptop computer, or tabletop computer. Cell sorting machines may also be fixed or mobile. Many such cell sorting machines are readily available commercially. The cell sorter machine may also be used in conjunction with suitable pre-treatment steps in various embodiments, e.g., a step to first remove certain cell fractions, e.g., red blood cell, using a device, a filter, or the like.

In some embodiments, the cell sorting machine may incorporate more than one detection stage, e.g., to provide negative or positive selection of cell types, to further improve isolation of cells, etc. For example, in some cases, antibodies may be used for detection. The antibodies may be free or attached to a magnetic particle, such as a nanoparticle. As a specific non-limiting example, antibodies for CD45 may be used to further isolate leukocytes from a pH-responsive isolate of cells in order to enhance the fraction of tumor cells in the isolated cell fraction. In some cases, the cells may be exposed to antibodies able to recognize a tumor-specific antigen, such as EpCAM, EphB4, HER2, EGFR, CEA, MUC-1, CD45, or other tumor-specific antigens known to those of ordinary skill in the art. In some cases, the antibodies are organ-specific antibodies, e.g., for determining the location of a tumor. For instance, the antibodies may be used for immunostaining purposes.

In another set of embodiments, determination of cells and/or sorting may occur using fluorescence microscopy. For instance, in one set of embodiments, the cells may be positioned slides, petri dishes, etc. for analysis using a fluorescence microscope. In some cases, this process may be automated or semi-automated. For example, a plurality of cells may be analyzed or automatically screened using a fluorescence microscope to determine which cells are fluorescent and which cells are not fluorescent (or are less fluorescent), e.g., as discussed herein. A person may analyze the fluorescence of the cells, or in some cases, the images may be analyzed using a computer programmed with appropriate image analysis techniques. Many such programs for image analysis of fluorescent samples are commercially available.

As another non-limiting example, separation may use antibodies able to recognize certain cell antigens. For example, the antibody may recognize cell antigens such as CD4, CD8, CD45, CD71, anti-eplison globin, or the like. As an example, the cells may be exposed to antibodies for fetal hemoglobin, thus separating fetal red blood cells from the initial isolated cell fraction based on factors such as pH. The remaining cells in that fraction may further be isolated in some cases using, for instance, CD4 and CD8 antibodies; for example, further selecting either cells negative for both CD4 and CD8, or positive for both CD4 and CD8 would provide for further isolation of fetal white cells. Negative selection for single positive cells (single positive or for CD4 or for CD8) also can be used in some cases. In some cases, the stem cells may be utilized for various purposes, e.g., for research or for therapeutic uses, etc.

It should be understood that in some cases, the devices or methods discussed herein may be fully or partially integrated into one or more larger devices, including human diagnostic equipment. It should also be understood that some embodiments may allow for measurement of many samples, e.g., either sequentially or simultaneously, and single experiments discussed herein are for convenience only and are not intended to be limiting.

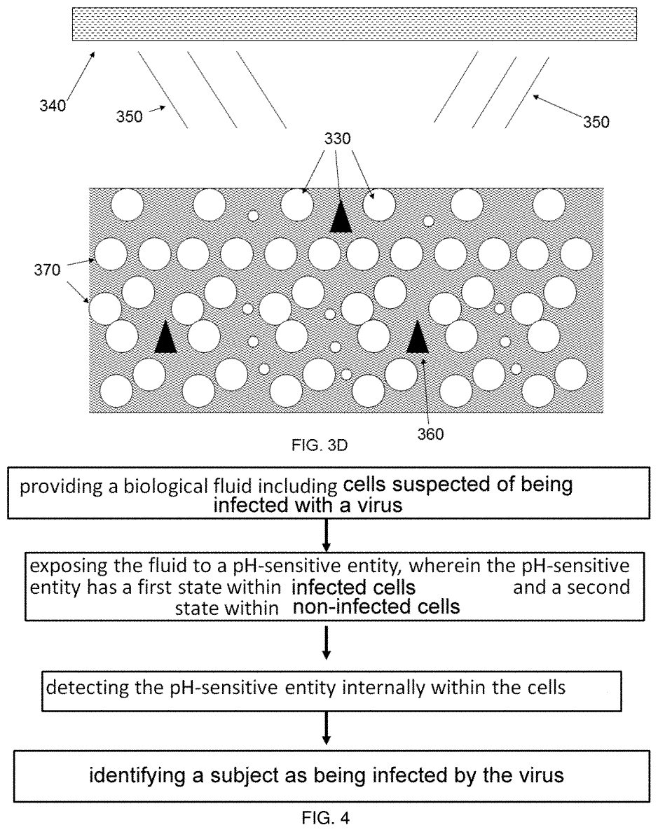

One example embodiment of the disclosure is now discussed with respect to FIG. 4 which shows a flowchart for a method, e.g., using cells potentially infected by a virus, such as a coronavirus. Although infected cells are discussed here, this is by way of example only, and other cells may also be used, e.g., any of the cells discussed herein.

In some cases, a biological fluid is used. The biological fluid may be, for example, blood, cerebral spinal fluid, urine, cervical fluid, nipple aspirate, saliva, phlegm, pleural or abdominal exudate, transudate, or the like. In another embodiment, the biological fluid is prepared from a homogenized tissue sample.

In some cases, additional techniques of using, analyzing, or characterizing the stem cells may be used, and those of ordinary skill in the art will be aware of a variety of techniques for using, manipulating, storing, analyzing, etc. stem cells. For example, the stem cells may be stored in some embodiments. The cells may be stored, for example, for future medical use. Any technique for storing or preserving cells known to those of ordinary skill in the art may be used. For example, the cells may be stored in a frozen state (e.g., the storing may include cryopreservation of stem cells. In some cases, the cells may be identified or isolated.

In some cases, for instance, a non-specific dye is added to a biological fluid. The dye may generally enter all cells, both those of interest (e.g., cells that are infected with a virus) as well as non-target cells (e.g., uninfected cells). Non-limiting examples of dyes include any of the ones discussed herein. As a non-limiting example, a dye may be used that is adapted to have a first property in a cytosolic pH that is basic, while the dye has a second property in a cytosolic pH that is neutral or acidic. In some cases, the dye may be fluorescent or otherwise identifiable in the higher pH environment internally of the cells, e.g., as discussed herein.

Once such target cells have been determined in some fashion (e.g., visualized), they may optionally be counted and/or collected. For example, collected cells, or the subject may be treated, e.g., to treat the infection.

In some cases, the pH-sensitive entity may be a dye or other entity discussed herein. For example, the pH-sensitive entity may be 2′,7′-bis(2-carboxyethyl)-5(6)-carboxyfluorescein tetrakis(acetoxymethyl) ester (BCECF AM), 5(6)-carboxy-2′,7′-dichlorofluorescein, 5(6)-carboxyfluorescein, 5(6)-carboxyfluorescein diacetate, 5(6)-carboxyfluorescein N-hydroxysuccinimide ester, 3,6-diacetoxyphthalonitrile, 6,8-dihydroxy-1,3-pyrenedisulfonic acid disodium salt, Eosin diacetate, or naphthofluorescein, etc.

As mentioned, in some embodiments, the pH-sensitive entity is fluorescent within the infected cells, and may be substantially less fluorescent in the non-infected cells. In some cases, the pH-sensitive entity may be fluorescent within the infected cells and is substantially less fluorescent in the non-infected cells. For example, in one set of embodiments, the pH-sensitive entity is fluorescent at a pH of greater than about 7.2 and substantially less fluorescent at a pH of less than about 7.

As mentioned, various embodiments of the present disclosure are generally directed to the determination and/or isolation of cells of interest from a population of cells. The volume of fluid containing the cells to be analyzed may be any suitable volume, for example, femtoliters, microliters, milliliters, liters, etc. In some cases, these cells may represent a very small part of the population of cells, as previously discussed. In certain embodiments, the cells may be determined, i.e., a population of cells is studied to identify whether certain cells are present, and/or how many of those cells are present. Thus, the determination may be qualitative and/or quantitative, in various applications. In certain embodiments, the cells of interest may be isolated from the population of cells. For example, these cells may be separated from the population of cells and placed at a first location (e.g., a collection chamber), while the other cells are placed at a second location (e.g., a second collection chamber), or perhaps discarded. In some cases, the isolated cells may be further analyzed, e.g., genetically, morphologically, cytopathologically, phenotypically, etc., e.g., as discussed herein. As a non-limiting example, the genetic analysis may include a search for genetic abnormalities such as chromosome defects.

As mentioned, in some embodiments, a cell sorting machine may be used, e.g., including any of those discussed herein. In some embodiments, the cell sorting machine may incorporate more than one detection stage, e.g., to provide negative or positive selection of cell types, to further improve isolation of cells, etc. For example, in some cases, antibodies may be used for detection. The antibodies may be free or attached to a magnetic particle, such as a nanoparticle. In addition, as another non-limiting example, an additional separation step may use antibodies able to recognize certain antigens. As an example, the cells may be exposed to antibodies to a virus, thus separating red blood cells from the initial isolated cell fraction based on pH.

It should be understood that in some cases, the devices or methods discussed herein may be fully or partially integrated into one or more devices, including human diagnostic equipment. It should also be understood that some embodiments could allow for measurement of many samples either sequentially or simultaneously, and single experiments discussed herein are for convenience only and are not intended to be limiting.

Thus, for example, certain aspects of the disclosure are generally directed to devices and methods for determining target cells of interest within a fluid, e.g., using the systems and methods as discussed herein. In some cases, an uncharged molecule is introduced into a fluid containing cells, such as blood or other fluids described herein), and the uncharged molecule is allowed to penetrate into the cells, e.g., through diffusion, osmosis, phagocytosis, or the like. Within at least some of the cells, the uncharged molecule may be converted into a charged form that does not readily exit the cells. The charged form may be determined within the device, e.g., using techniques such as fluorescence, radioactivity or nuclear particle release (e.g., detection of gamma rays, beta particles, and/or alpha particles, etc.), color changes, energy release, electromagnetic radiation release, chemical reactivity, biological reactivity, chemical polarity, solubility changes, or the like.

In another aspect of the device, the cell selector is realized as a cell cytometer or microscope adapted to able to count cells. In another aspect of the device, the cell cytometer or microscope is adapted to isolate and/or sort cells. In another aspect of the device, the fluid is selected from blood, blood serum, cerebral spinal fluid, urine, cervical fluid, nipple aspirate, saliva, phlegm, pleural or abdominal exudate or transudate.

Without being bound by any theory, the following discussion is offered to provide greater insight into certain embodiments, e.g., where a signaling molecule, or other entity, is introduced into cells. The present disclosure, in some embodiments thereof, relates to methods and devices for delivering a charged compound into a cytosol or organelle of a cell. In some embodiments, an uncharged molecule or compound is allowed to enter cells in a sample; in cells of interest does the uncharged molecule undergo a chemical or biochemical transformation to at least one charged molecule that cannot leave the cell which it entered. For example, the uncharged molecule or compound may become charged through a change in pH, interaction with an enzyme such as an esterase or a protease, or the like, e.g., as discussed herein. Non-limiting examples of potentially suitable uncharged molecules are well-known. The charged molecule may have desirable properties including, but not limited to, fluorescence or therapeutic action on the cell in which it is located. In a non-target cell, for example, the uncharged molecule may remain in its original state and generally has neither properties for identification or therapeutic action. For instance, in some cases, in the non-target cell, less than about 30%, less than about 25%, less than about 20%, less than about 15%, less than about 10%, or less than about 5% of the uncharged molecule is converted into a charged state.

It is well known that charged molecules generally do not pass through cellular membranes, unless they are brought into a cell, e.g., via a specific transfer mechanism. This barrier to entry may present a challenge to delivering either biologically active treatments or specific probes to cells. On the other hand, uncharged molecules, especially those with extended pi rings, can often pass through hydrophobic membrane barriers.

Some embodiments of the disclosure describes phenomena related to trapping charged molecules in a predetermined cell or group of cells based on cell-specific chemical or other activities that are adapted to convert uncharged molecules into charged molecules unable to escape the target cell(s). For example, it is understood that a plurality of signaling molecules may be employed to enter either target and/or non-target cell types. In some embodiments, non-target cells may be targeted with signaling material, while target cells lack a specific activity to transform the uncharged compound into a charged compound that may readily be identified through color, fluorescence or other appropriate techniques. In other embodiments, however, the opposite is true, e.g., target cells may be targeted with signaling material, while non-target cells lack a specific activity to transform the uncharged compound into a charged compound.

Certain embodiments of the instant disclosure have been show discrimination of target to non-target cells at ratios of at least about 1:10, at least about 1:100, at least about 1:1,000, at least about 1:10,000, at least about 1:100,00, at least about 1:1,000,000, or higher in some cases.

Some embodiments can distinguish alkaline from acidic cells, e.g., where target cells are alkaline internally and non-target cells are acidic or neutral, or vice versa. Additionally and/or alternatively, embodiments can allow for detection of target cells from non-target cells, wherein target cells include acidic intern pH values and non-target cells show neutral or alkaline pH readings in cellular spaces.

While pH-sensitive entities were described above, it should be understood that other uncharged molecules or compounds may also be used in other embodiments as signaling entities, in addition to or instead of the above-described pH-sensitive entities. For example, the charged compound may be detected using, for example, radioactivity, color, fluorescence, energy release, chemical reactivity, biological reactivity, chemical polarity, or solubility, e.g., using an appropriate signaling entity. Non-limiting examples of such compounds are given in Table 1.

In some cases, the unchanged molecule or compound may be generally polar solvent soluble so as to allow its dissolution in a liquid which may be added directly or after water dilution to a fluid containing cells. For example, the polar solvent may be water, DMSO, or other suitable polar solvents. Without wishing to be bound by any theory, it is believed that the lack of charge facilitates movement of uncharged molecule through an outer membrane of a cell and in some embodiments through additional membranes of organelles such as those associated with mitochondria and nuclei.

Once entered a cell, the molecule may be allowed to remain within the cells, e.g., to be converted into a charged form. The uncharged molecule may become charged within the cell, or within a specific region or portion of the cell, e.g., in the cytoplasm, within an organelle such as a mitochondrion, etc. For example, the compound may become charged at a pH of a cytosol of a target cell. The charged state may be positive or negative.

In some embodiments, the compound, when charged, may exhibit anti-cancer, anti-bacterial, or anti-inflammatory behavior. However, in some cases, the compound may have relatively low therapeutic effects when in an uncharged state, although this is not a requirement. The uncharged compound may be generally solvent in water, polar solvents or water-polar solvent mixtures. For example, a protease inhibitor may be in an inactive form when part of an uncharged molecule adapted to pass through cellular membranes; when the uncharged molecule is chemically or biochemically converted to a charged molecule, the charged molecule may be converted into an active protease inhibitor.

In some cases, the compound may generally diffuse or be actively transported through a cellular membrane when in an unchanged state. The compound may be polar in some embodiments, and/or the compound may include internal charges that cancel to yield a net charge of zero. In some cases, the compound may be zwitterionic or may be fully or partially charged under some conditions.

The time (e.g., “incubation time”) may be anywhere between a few minutes to hours or days. For example, time may be given to allow the uncharged molecule to enter as many of the cells in a sample as possible. For example, the time may be half an hour or less, one hour or less, two hours or less, three hours or less, four hours or less, six hours or less, twelve hours or less, or twenty-four hours, or less. More than twenty-four hours is also possible in some cases. The conversion of the uncharged compound into a charged compound (or more than one charged compound in some cases) generally occurs in the target cell. In some cases, this may occur without any manipulation and/or energy addition from an external source.

Conditions such as pH or presence of specific catalytic entities within a target cell may be used to drive the conversion from an unchanged state to a charged state. In some cases, such conditions may not occur in non-target cells, or may occur, but at at very low rates or concentrations so as to make the product insignificant, or at least distinguishable with respect to the target cells.

A non-limiting example of a condition within a target cell that may be different from a non-target cell is pH. Examples of pH differences include any of those provided herein. In some cases, for example, an uncharged molecule may become charged at a basic pH value, e.g., a value higher than about 7.0, higher than about 7.5, higher than about 8.0, etc. In some embodiments, an uncharged molecule may become charged at an acidic pH value, e.g., a value less than about 7.0, less than about 6.5, less than about 6.0, etc.

However, the disclosure is not limited to only pH or pH changes. In another set of embodiments, an enzyme or a metallic catalyst may be able to convert an uncharged molecule into a charged molecule. For example, the enzyme may be an esterase or a protease. Non-limiting examples of specific molecules that can be altered using enzymes or catalysts (e.g., metal catalysts) can be seen in Table 1.

TABLE 1

| Name/chemical name | Structure |
|---|---|
| Azactidine 4-amino-1-((2R,3R,4S,5R)- 3,4-dihydroxy-5- (hydroxymethyl) tetrahydrofuran-2-yl)- 1,3,5-triazin-2(1H)-one | 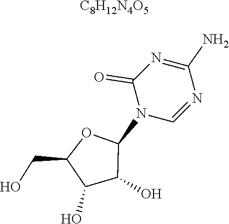 [SMILES: "C8H12N4O5" ; Nc1ncn([C@@H]2O[C@H](CO)[C@@H](O)[C@H]2O)c(=O)n1] |
| Bendamistine (Cl) 4-[5-[bis(2-chloroethyl) amino]-1- methylbenzimidazol-2- yl]butanoic acid hydrochloride | 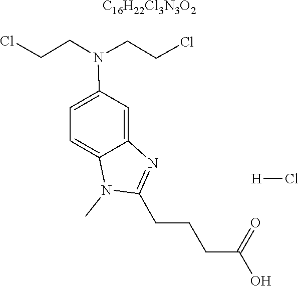 |
| Bustafan butan-1,4-diyl dimethanesulfonate | 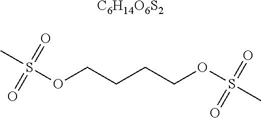 |
| Carmustin | 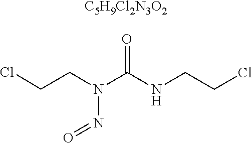 [SMILES: O=NN(CCCl)C(=O)NCCCl ; "C5H9Cl2N3O2"] |
| Carboplatin | 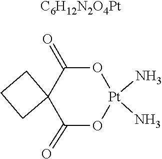 |
| Chlorambucil 4-(4-(bis(2- chloroethyl)amino) phenyl)butanoic acid | 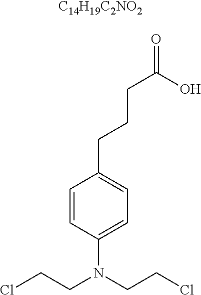 [SMILES: "C14H19C2NO2" ; O=C(O)CCCc1ccc(N(CCCl)CCCl)cc1] |
| Cyclophosphamide 2-[bis(2-chloroethyl) amino]tetrahydro- 2H-1,3,2- oxazaphosphorine-2- oxide monohydrate | 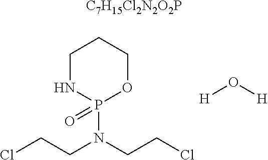 |
| Dacarbazine (E)-5-(3,3-dimethyltriaz-1- en-1-yl)-1H-imidazole-4- carboxamide | 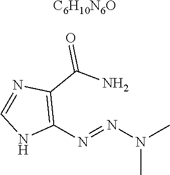 |
| Diaziquone diethyl (2,5-di(aziridin- 1-yl)-3,6- dioxocyclohexa-1,4-diene- 1,4-diyl)dicarbamate | 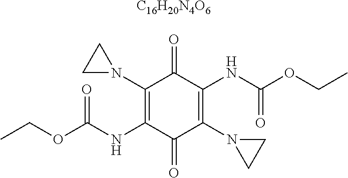 [SMILES: "C16H20N4O6" ; CCOC(=O)NC1=C(N2CC2)C(=O)C(NC(=O)OCC)=C(N2CC2)C1=O] |
| Ifosfamide 3-(2-chloroethyl)-2- ((2-chloroethyl) amino)-1,3,2- oxazaphosphinane 2-oxide | 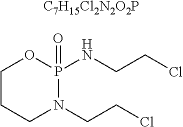 |
| Melphalan Hydrochloride (S)-2-amino- 3-(4-(bis(2- chloroethyl)amino) phenyl)propanoic acid hydrochloride | 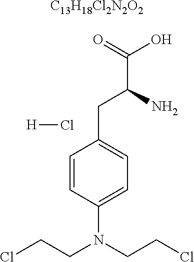 |
| Methylisoindigotin (E)-1,1′-dimethyl-[3,3′- biindolinylidene]- 2,2′-dione | 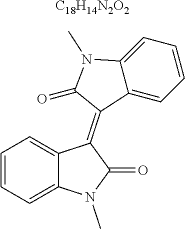 |
| Procarbazine N-isopropyl-4-[(2- methylhydrazino) methyl]benzamide | 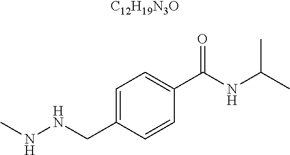 [SMILES: "C12H19N3O" ; CNNCc1ccc(C(=O)NC(C)C)cc1] |
| Streptozocin 1-methyl-1-nitroso-3- ((3R,4R,5S,6R)-2,4,5- trihydroxy-6- (hydroxymethyl) tetrahydro- 2H-pyran-3-yl)urea | 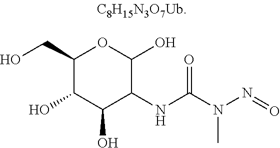 [SMILES: CN(N=O)C(=O)NC1C(O)O[C@H](CO)[C@@H](O)[C@@H]1O] |
| Temozolomide 3-methyl-4-oxo-3,4- dihydroimidazo[5,1-d] [1,2,3,5]tetrazine- 8-carboxamide. | 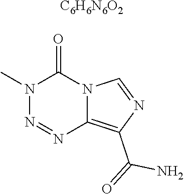 |
| Pemetrexed Disodium sodium (S)-2-(4-(2-(2- amino-4-oxo-4,7- dihydro-1H- pyrrolo[2,3-d]pyrimidin- 5-yl)ethyl)benzamido) pentanedioate heptahydrate. | 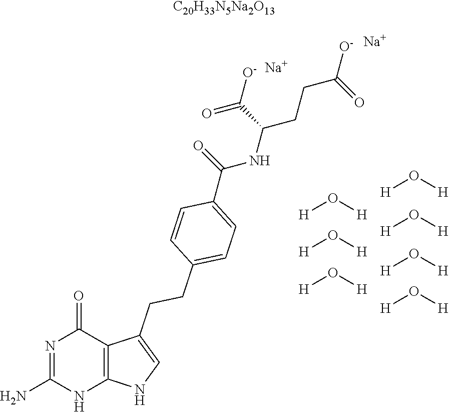 |
| Raltitrexed (S)-2-(5-(methyl((2- methyl-2-oxo- 1,4-dihydroquinazolin-6- yl)methyl)amino)thiophene- 2-carboxamido) pentanedioc acid | 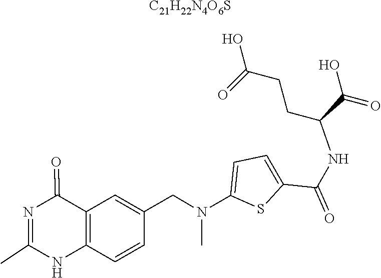 [SMILES: "C21H22N4O6S" ; Cc1nc(=O)c2cc(CN(C)c3ccc(C(=O)N[C@@H](CCC(=O)O)C(=O)O)s3)ccc2[nH]1] |
| Capecitabine pentyl (1-((2R,3R,4S,5R)- 3,4-dihydroxy-5- methyltetrahydrofuran-2-yl)- 5-fluoro-2-oxo-1,2- dihydropyrimidin-4-yl) carbamate | 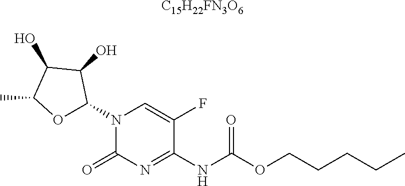 [SMILES: "C15H22FN3O6" ; CCCCCOC(=O)Nc1nc(=O)n([C@@H]2O[C@H](C)[C@@H](O)[C@H]2O)cc1F] |
| Cytarabine 4-amino-1-[(2R,3S,4R,5R)- 3,4-dihydroxy-5- (hydroxymethyl)oxolan-2-yl] pyrimidin-2-one | 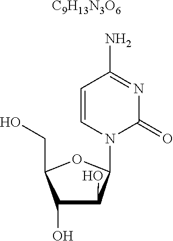 |
| 5-Fluoro Uracil 5-fluoro-1H,3H- pyrimidine-2,4-dione | 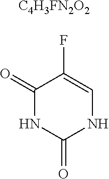 |
| Fludarabine phosphate ((2R,3S,4S,5R)-5-(6-amino- 2-fluoro-9H-purin-9-yl)-3,4- dihydroxytetra- hydrofuran-2-yl) methyl dihydrogen phosphate | 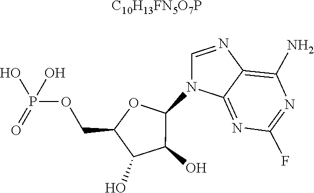 [SMILES: "C10H13FN5O7P" ; Nc1nc(F)nc2c1ncn2[C@@H]1O[C@H](COP(=O)(O)O)[C@@H](O)[C@@H]1O] |
| Gemcitabine hydrochloride 4-amino-1-((2R,4R,5R)-3,3- difluoro-4-hydroxy-5- (hydroxymethyl) tetrahydrofuran- 2-yl)pyrimidin-2(1H)-one hydrochloride | 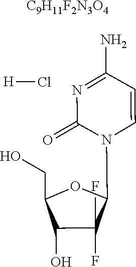 |
| Methotrexate (S)-2-(4-(((2,4- diaminopteridin-6- yl)methyl)(methyl)amino) benzamido)pentanedioic acid. | 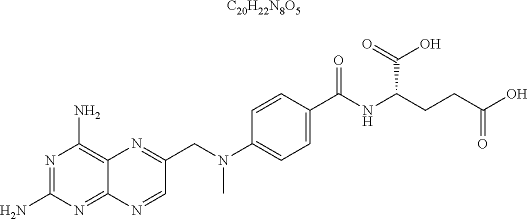 [SMILES: "C20H22N8O5" ; CN(Cc1cnc2nc(N)nc(N)c2n1)c1ccc(C(=O)N[C@@H](CCC(=O)O)C(=O)O)cc1] |
| Pemetrexed disidum sodium (S)-2-(4-(2-(2- amino-4-oxo-4,7- dihydro-1H- pyrrolo[2,3-d]pyrimidin-5- yl)ethyl)benzamido) pentanedioate heptahydrate. | 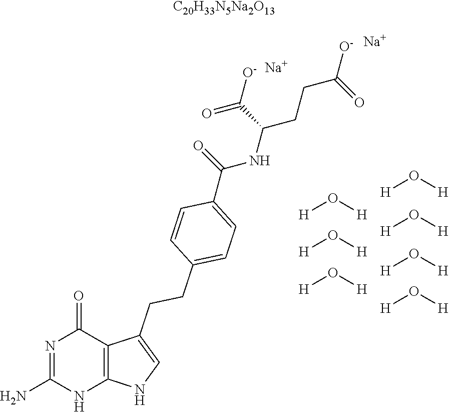 |
| LY294002 2-(4-morphinyl)-8-phenyl- 4H-1-benzopyran-4-one | 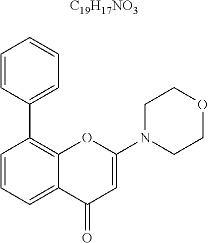 [SMILES: "C19H17NO3" ; O=c1cc(N2CCOCC2)oc2c(-c3ccccc3)cccc12] |
| Deguelin (7aS,13aS)-13,13a-Dihydro- 9,10-dimethoxy- 3,3-dimethyl- 3H-bis[1]benzopyrano [3,4-b:6′,5′-e]pyran- 7(7aH)-one | 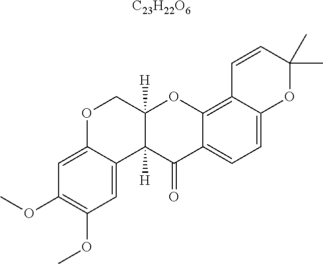 |
| Raltitrexed (S)-2-(5-(methyl((2-methyl- 4-oxo-1,4- dihydroquinazolin-6- yl)methyl)amino) thiophene-2- carboxamido) pentanedioic acid | 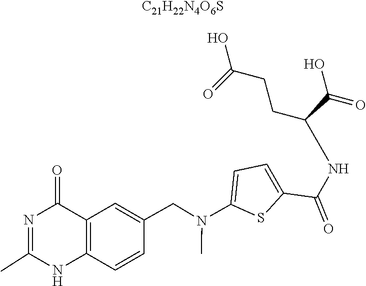 [SMILES: "C21H22N4O6S" ; Cc1nc(=O)c2cc(CN(C)c3ccc(C(=O)N[C@@H](CCC(=O)O)C(=O)O)s3)ccc2[nH]1] |
| Dactinomycin 2-amino-N1,N9-bis(6,13- diisopropyl-2,5,9-trimethyl- 1,4,7,11,14- pentaoxohexadecahydro- 1H-pyrrolo[2,1- i][1,4,7,10,13] oxatetraazacyclo- hexadecin-10-yl)-4,6- dimethyl-3-oxo-3H- phenoxazine- 1,9-dicarboxamide | 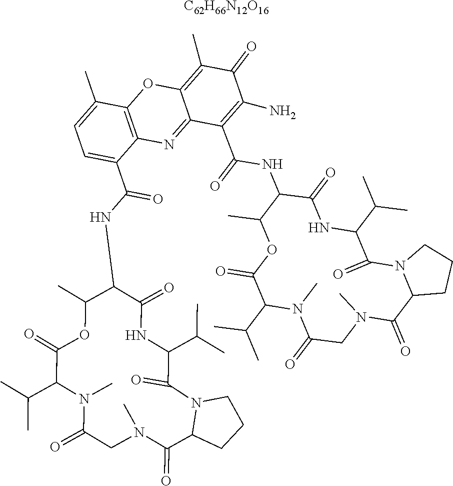 |
| Bleomycin 3-[[2-[2-[2-[2- [4-[2-[6-Amino-2- [1-(2-amino-2-carbamoyl- ethyl)amino-2-carbamoyl- ethyl]-5-methyl-pyrimidin- 4-yl]carbonylamino-3- [3-[4-carbamoyloxy-3,5- dihydroxy-6- (hydroxymethyl) tetrahydropyran-2-yl]oxy- 4,5-dihydroxy-6- (hydroxymethyl) tetrahydropyran-2-yl]oxy- 3-(3H-imidazol-4-yl) propanoyl]amino-3- hydroxy-2-methyl- pentanoyl]amino-3- hydroxy-butanoyl] aminoethyl]-1,3-thiazol- 4-yl]-1,3-thiazol-4-yl] carbonylamino]propyl- dimethyl-sulfonium hydrogen sulfate | 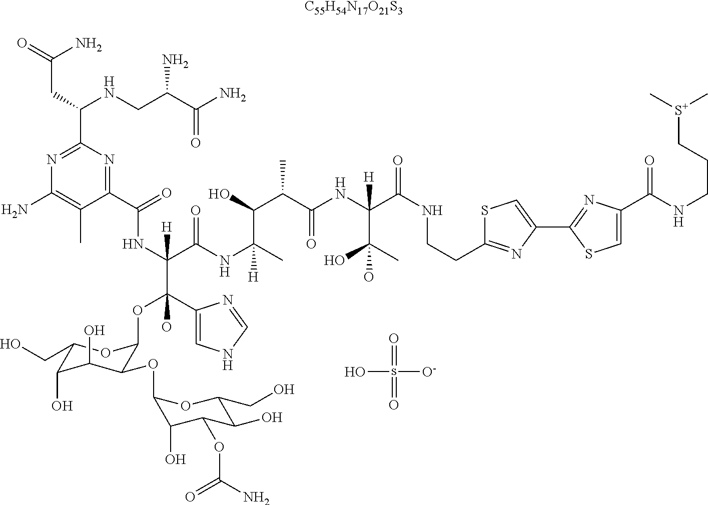 |
| Daunorubicin hydrochlorid (8S-cis)-8-Acetyl-10- ((3-amino-2,3,6-trideoxy- alpha-L-lyxo- hexopyranosyl)oxy)- 7,8,9,10-tetrahydro- 6,8,11-trihydoxy-1- methoxy-5,12- naphthacenedione hydrochloride | 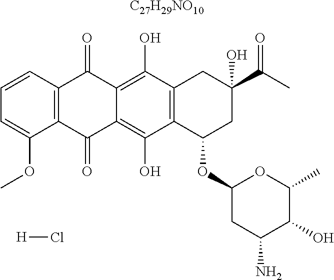 [SMILES: "C27H29NO10" ; COc1cccc2c1C(=O)c1c(O)c3c(c(O)c1C2=O)C[C@@](O)(C(C)=O)C[C@@H]3O[C@@H]1C[C@@H](N)[C@@H](O)[C@@H](C)O1 ; Cl] |
| Doxorubicin (8S,10S)-10-(((2R,4S,5S, 6S)-4-amino-5-hydroxy- 6-methyltetrahydro-2H- pyran-2-yl)oxy)-6,8,11- trihydroxy-8-(2- hydroxyacetyl)-1- methoxy-7,8,9,10- tetrahydrotetracene-5,12- dione hydrochloride | 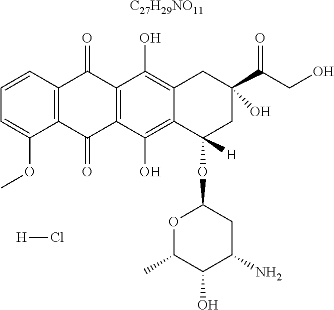 [SMILES: "C27H29NO11" ; COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1 ; Cl] |
| Epirubicin (8S,10S)-10-(((2R,4S,5R, 6S)-4-amino-5-hydroxy- 6-methyltetrahydro- 2H-pyran-2-yl)oxy)-6,8,11- trihydroxy-8-(2- hydroxyacetyl)-1- methoxy-7,8,9,10- tetrahydrotetracene-5,12- dione hydrochloride | 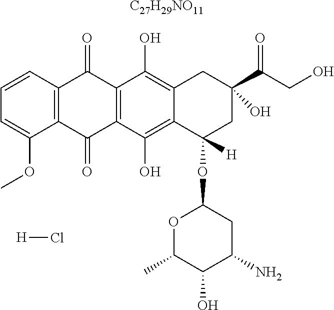 [SMILES: "C27H29NO11" ; COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1 ; Cl] |
| Idarubicin (7S,9S)-9-acetyl-7- (((2R,4S,5S,6S)-4-amino- 5-hydroxy-6- methyltetrahydro-2H- pyran-2-yl)oxy)-6,9,11- trihydroxy-7,8,9,10- tetrahydrotetracene-5,12- dione hydrochloride | 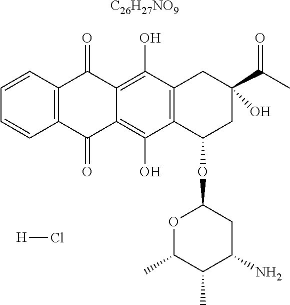 [SMILES: "C26H27NO9" ; CC(=O)[C@]1(O)Cc2c(O)c3c(c(O)c2[C@@H](O[C@H]2C[C@H](N)[C@H](C)[C@H](C)O2)C1)C(=O)c1ccccc1C3=O ; Cl] |
| Mitomycin ((1aS,8S,8aR,8bS)-6- amino-8a-methoxy-5- methyl-4,7-dioxo-1,1a,2,4, 7,8,8a,8b- octahydroazirino [2′,3′:3,4]pyrrolo[1,2- a]indol-8-yl)methyl carbamate | 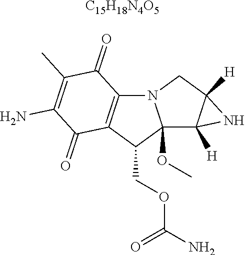 |
| Mitoxantrone hydrochlorid 1,4-dihydroxy-5,8-bis((2- ((2-hydroxyethyl) amino)ethyl)amino) anthracene-9,10-dione dihydrochloride | 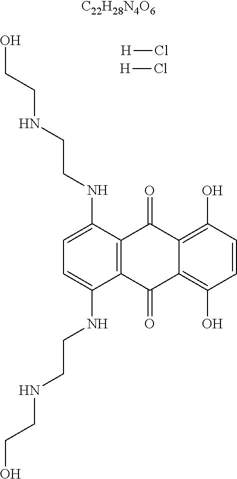 |
| Etoposide (5R,5aR)-9-(((2R,4aR,6R, 7R,8R,8aS)-7,8- dihydroxy-2- methylhexahydropyrano [3,2-d]dioxin-6-yl) oxy)-5-(4-hydroxy- 3,5-dimethoxyphenyl)- 5,5a,8a,9- tetrahydrofuro [3′,4′:6,7]naphtho[2,3-d] [1,3]dioxol-6(8H)-one | 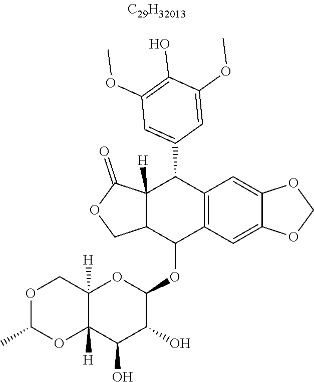 [SMILES: COc1cc([C@@H]2c3cc4c(cc3C(O[C@@H]3O[C@@H]5CO[C@@H](C)O[C@H]5[C@H](O)[C@H]3O)C3COC(=O)[C@@H]32)OCO4)cc(OC)c1O] |
| Irinotecan hydrochloride (S)-4,11-diethyl-4- hydroxy-3,14-dioxo- 3,4,12,14-tetrahydro- 1H-pyrano[3′,4′:6,7] indolizino[1,2-b] quinolin-9-yl [1,4′- bipiperidine]-1′- carboxylate hydrochloride trihydrate. | 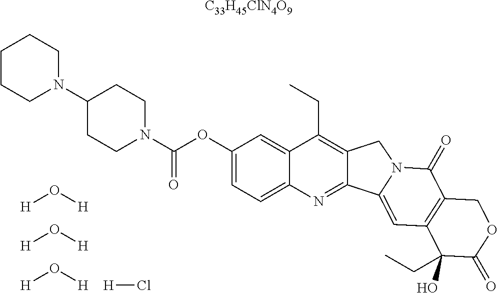 |
| Palcitaxel (2aR,4S,4aS,6R,9S,11S, 12S,12aR,12bS)-9- (((2R,3S)-3- benzamido-2-hydroxy- 3-phenylpropanoyl) oxy)-12-(benzyloxy)- 4,11-dihydroxy- 4a,8,13,13-tetramethyl-5- oxo-2a,3,4,4a,5,6,9,10,11, 12,12a,12b-dodecahydro- 1H-7,11-methanocyclo- deca[3,4]benzo[1,2-b] oxete-6,12b-diyl diacetate | 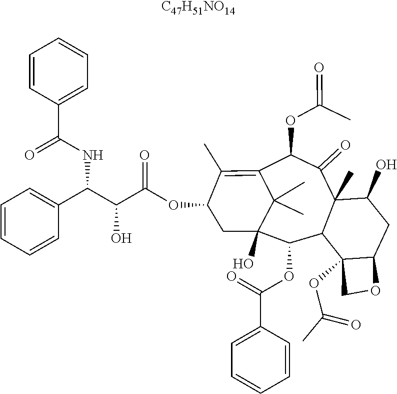 [SMILES: "C47H51NO14" ; CC(=O)O[C@H]1C(=O)[C@@]2(C)C([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O] |
| Topotecan (S)-10-((dimethylamino) methyl)-4-ethyl-4,9- dihydroxy-1H-pyrano [3′,4′:6,7]indolizino[1,2- b]quinoline-3,14- (4H,12H)-dione hydrochloride | 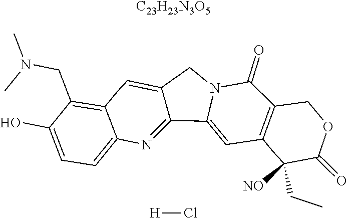 |
| Vinblasine (3aR,3a1R,4R,5aS,10bR)- methyl 4-acetoxy-3a- ethyl-9-((5S,7R,9S)-5- ethyl-hydroxy-9- (methoxycarbonyl)- 2,4,5,6,7,8,9,10- octahydro-1H-3,7- methano[1] azacycloundecino[5,4-b] indol-9-yl)-5-hydroxy-8- methoxy-6-methyl- 3a,3a1,4,5,5a,6,11,12- octahydro-1H- indolizino[8,1-cd] carbazole-5- carboxylate sulfate | 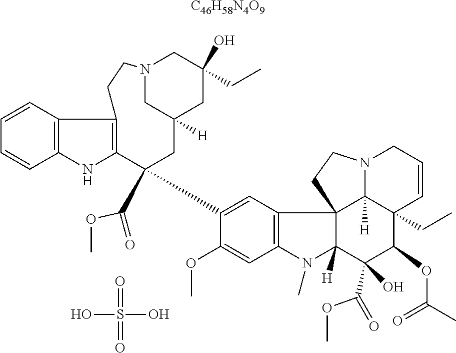 |
| Vincristine sulfate (3aR,3a1R,4R,5S,5aR, 10bR)-methyl 4- acetoxy-3a-ethyl-9- ((3S,5S,7S,9S)-5-ethyl- 5-hydroxy-9- (methoxycarbonyl)- 2,4,5,6,7,8,9,10- octahydro-1H-3,7- methano[1]azacyclo- undecino[5,4-b] indol-9-yl)-6-formyl-5- hydroxy-8-metboxy- 3a,3a1,4,5,5a,6,11,12- octahydro-1H- indolizino[8,1-cd] carbazole-5- carboxylate sulfate. | 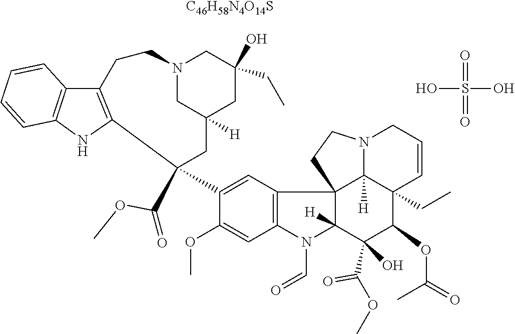 |
| Vinorelbine tartrate (3aR,3a1R,4R,5S,5aR, 10bR)-methyl 4-acetoxy- 3a-ethyl-9-((2R,8S)-4- ethyl-8-(methoxy- carbonyl)-1,3,6,7,8,9- hexahydro-2,6- methanoazecino[4,3-b] indol-8-yl)-5-hydroxy-8- methoxy-6-methyl-3a,3a1, 4,5,5a,6,11,12-octahydro- 1H-indolizino[8,1-cd] carbazole-5-carboxylate bis((2R,3R)-2,3- dihydroxysuccinate) | 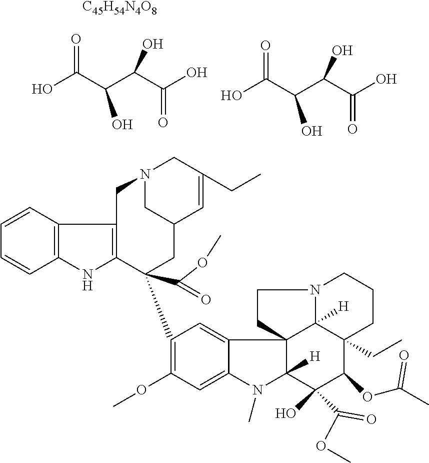 |
| Carboplatin platinum, diammine[1,1- cyclobutanedicarboxylate (2o)-O,O′]-, (SP-4-2) | 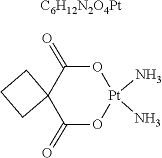 |
| Cisplatin (SP-4-2)-diamminedi- chloroplatinum; platinum, diaminedichloro-, cis- (8CI). | 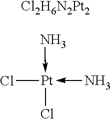 |
| Oxaliplatin [(1R,2R)-cyclohexane-1,2- diamine](ethanodioato- O,O′)platinum(II) | 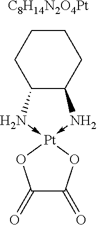 |
| Ametantrone 1,4-bis((2-((2-hydroxy- ethyl)amino)ethyl) amino)anthracene- 9,10-dione | 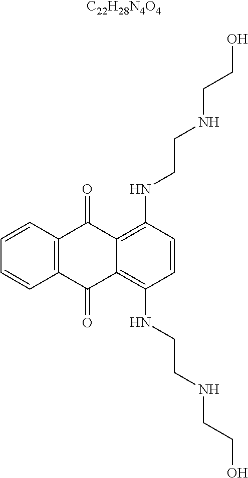 [SMILES: "C22H28N4O4" ; O=C1c2ccccc2C(=O)c2c(NCCNCCO)ccc(NCCNCCO)c21] |
| Apaziquone 5-(aziridin-1-yl)-3- (hydroxymethyl)-2-[(E)- 3-hydroxyprop-1-enyl]- 1-methylindole-4,7-dione | 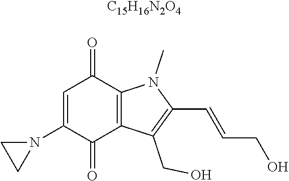 |
| Bromcresol Green | 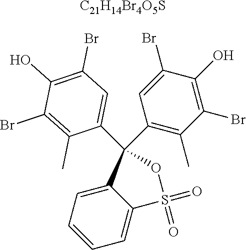 |
| Bevacizumab | 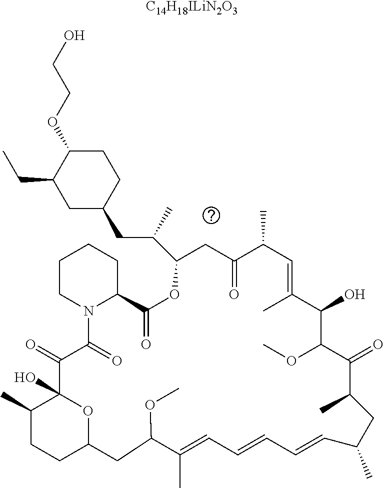 [SMILES: "C14H18ILiN2O3" ; CC[C@@H]1C[C@H](C[C@H](C)[C@@H]2CC(=O)[C@H](C)/C=C(\C)[C@@H](O)C(OC)C(=O)[C@H](C)C[C@H](C)/C=C/C=C/C=C(\C)C(OC)CC3CC[C@@H](C)[C@@](O)(O3)C(=O)C(=O)N3CCCC[C@H]3C(=O)O2)CC[C@H]1OCCO] |
| Erlotinib hydrichlorid N-(3-Ethynylphenyl)- 6,7-bis(2-methoxyethoxy)- 4-quinazolinamine Monohydrochloride. | 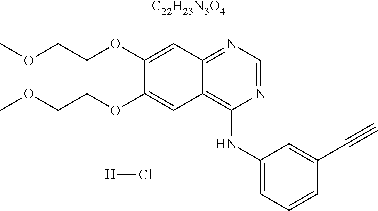 |
| Geftinib N-(3-chloro-4- fluorophenyl)-7- methoxy-6-(3- morpholinopropoxy) quinazolin-4-amine | 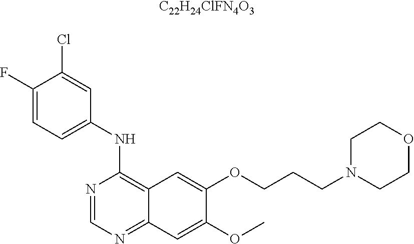 |
| Imatinib mesylate N-(4-methyl-3-((4- (pyridin-3-yl)pyrimidin-2- yl)amino)phenyl)-4-((4- methylpiperazin-1- yl)methyl)benzamide methanesulfonate | 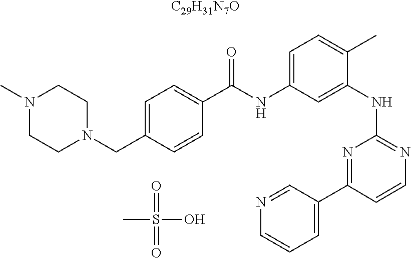 |
| Lapatinib N-(3-chloro-4-((3- fluorobenzyl)oxy) phenyl)-6-(5-(((2- (methylsulfonyl) ethyl)amino)methyl) furan-2-yl) quinazolin-4-amine | 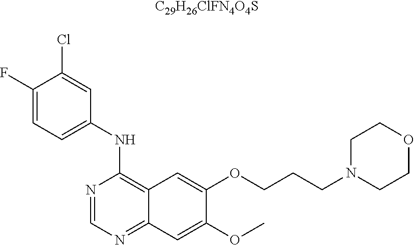 [SMILES: "C29H26ClFN4O4S" ; COc1cc2ncnc(Nc3ccc(F)c(Cl)c3)c2cc1OCCCN1CCOCC1] |
| Sunitinib malate (Z)-N-(2-(diethylamino) ethyl)-5-((5-fluoro-2- oxoindolin-3-ylidene) methyl)-2,4-dimethyl-1H- pyrrole-3- carboxamide | 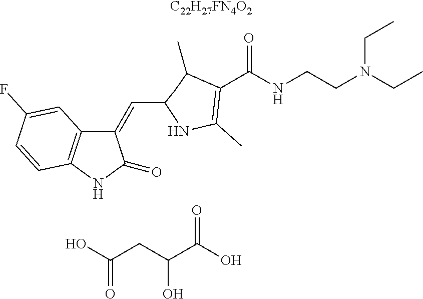 |
| Saracatinib N-(5-Chloro-1,3- benzodioxol-4-yl)-7-[2- (4-methyl-1- piperazinyl)ethoxy]-5- [(tetrahydro-2H-pyran- 4-yl)oxy]-4- quinazolinamine | 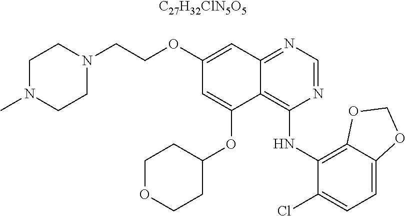 |
| Sorafenib 4-(4-(3-(4-chloro-3- (trifluoromethyl)phenyl) ureido)phenoxy)- N- methylpicolinamide 4- methylbenzenesulfonate. | 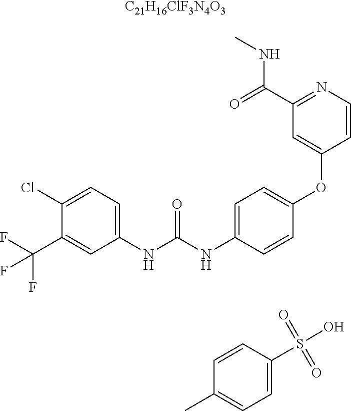 [SMILES: "C21H16ClF3N4O3" ; CNC(=O)c1cc(Oc2ccc(NC(=O)Nc3ccc(Cl)c(C(F)(F)F)c3)cc2)ccn1 ; Cc1ccc(S(=O)(=O)O)cc1] |
| Tozasertib (N-[4-({4-(4-methyl- piperazin-1-yl)-6-[(3- methyl-1H-pyrazol-5- yl)amino]pyrimidin-2- yl}thio)phenyl] cyclopropanecarboxamide) | 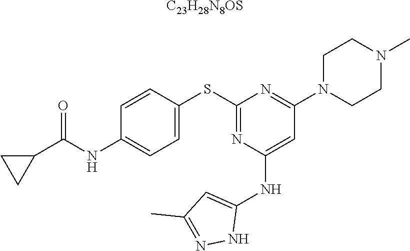 [SMILES: "C23H28N8OS" ; Cc1cc(Nc2cc(N3CCN(C)CC3)nc(Sc3ccc(NC(=O)C4CC4)cc3)n2)[nH]n1] |
| Plerixafor 1,4-bis((1,4,8,11- tetraazacyclotetradecan-1- yl)methyl)benzene | 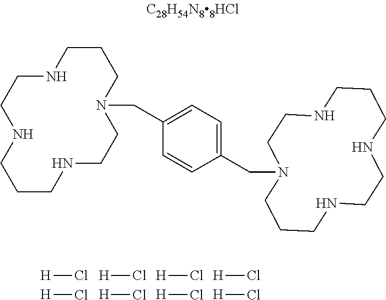 |
| Temsirolimus (1R,2R,4S)-4-{(2R)-2- [(3S,6R,7E,9R,10R, 12R,14S,15E,17E,19E, 21S,23S,26R,27R,34aS)- 9,27-dihydroxy-10,21- dimethoxy-6,8,12,14,20, 26-hexamethyl-1,5,11,28, 29-pentaoxo-1,4,5,6,9, 10,11,12,13,14,21,22,23, 24,25,26,27,28,29,31,32, 33,34,34a-tetracosahydro- 3H-23,27-epoxypyrido[2,1- c][1,4]oxazacyclohentria- contin-3-yl]propyl}-2- methoxycyclohexyl 3- hydroxy-2-(hydroxy- methyl)-2-methyl- propanoate | 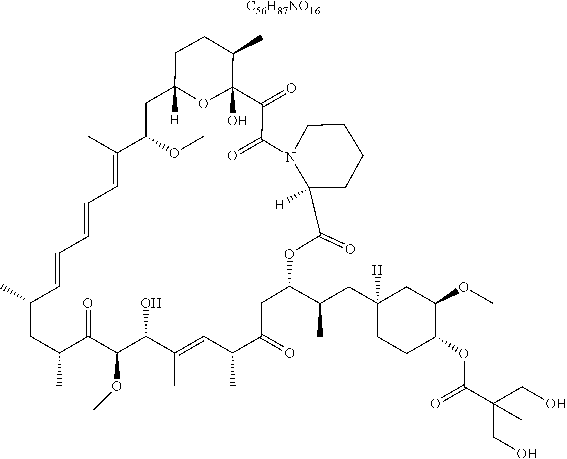 [SMILES: "C56H87NO16" ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)C(C)(CO)CO)[C@H](OC)C2)CC(=O)[C@H](C)/C=C(\C)[C@@H](O)[C@@H](OC)C(=O)[C@H](C)C[C@H](C)/C=C/C=C/C=C/1C] |
| Clodronate (2S,3S,4R,5R)-5-(6- amino-2-chloro-9H- purin-9-yl)-4-fluoro- 2-(hydroxymethyl) tetrahydrofuran-3-ol | 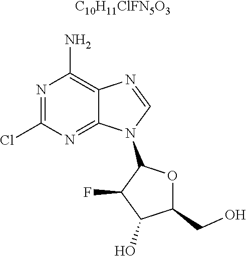 |
| Anastrozole 2,2′-(5-((1H-1,2,4-triazol- 1-yl)methyl)-1,3- phenylene)bis(2- methylpropanenitrile) | 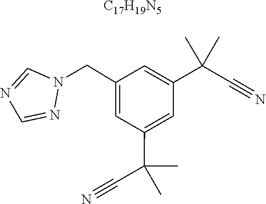 [SMILES: "C17H19N5" ; CC(C)(C#N)c1cc(Cn2cncn2)cc(C(C)(C)C#N)c1] |
| Abiraterone (3S,8R,9S,10R,13S,14S)- 10,13-dimethyl-17- (pyridin-3-yl)-2,3,4,7,8, 9,10,11,12,13,14,15- dodecahydro-1H- cyclopenta[a] phenanthren-3-ol. | 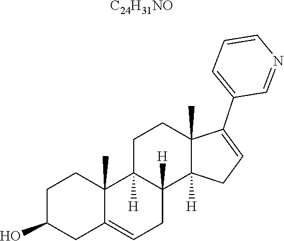 |
| Bexarotene 4-(1-(3,5,5,8,8-pentamethyl- 5,6,7,8-tetrahydro- naphthalen-2-yl)vinyl) benzoic acid | 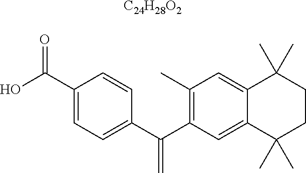 [SMILES: "C24H28O2" ; C=C(c1ccc(C(=O)O)cc1)c1cc2c(cc1C)C(C)(C)CCC2(C)C] |
| Bicalutamide N-(4-cyano-3-(trifluoro- methyl)phenyl)-3-((4- fluorophenyl)sulfonyl)-2- hydroxy-2- methylpropanamide | 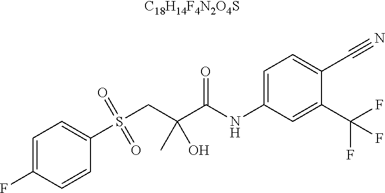 [SMILES: "C18H14F4N2O4S" ; CC(O)(CS(=O)(=O)c1ccc(F)cc1)C(=O)Nc1ccc(C#N)c(C(F)(F)F)c1] |
| Buserelin 1-(3-((1H-imidazol-4-yl) methyl)-6-((1H-indol-3-yl) methyl)-15-(tert-butoxy- methyl)-21-(3- guanidinopropyl)-12-(4- hydroxybenzyl)-9- (hydroxymethyl)-18- isobutyl-1,4,7,10,11,13, 16,19-heptaoxo-1-(5- oxopyrrolidin-2-yl)- 2,5,8,11,14,17,20- heptaazadocosan-22-oyl)-N- ethylpyrrolidine-2- carboxamide | 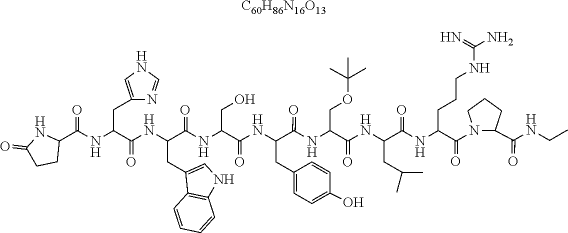 [SMILES: "C60H86N16O13" ; CCNC(=O)C1CCCN1C(=O)C(CCCNC(=N)N)NC(=O)C(CC(C)C)NC(=O)C(COC(C)(C)C)NC(=O)C(Cc1ccc(O)cc1)NC(=O)C(CO)NC(=O)C(Cc1c[nH]c2ccccc12)NC(=O)C(Cc1c[nH]cn1)NC(=O)C1CCC(=O)N1] |
| Degarelix acetate (S)-N-(4-((2S,5S,8R, 11R,14R)-2-(((R)-1- (((S-1-(((S)-1-((S)-2- (((R)-1-amino-1- oxopropan-2-yl) carbamoyl)pyrrolidin-1- yl)-6-(isopropylamino)-1- oxohexan-2-yl)amino)-4- methyl-1-oxopentan-2- yl)amino)-1-oxo-3-(4- ureidophenyl)propan-2- yl)carbamoyl)-11-(4- chlorobenzyl)-5- (hydroxymethyl)-14- (naphthanlen-2-ylmethyl)- 4,7,10,13,16-pentaoxo-8- (pyridin-3-ylmethyl)- 3,6,9,12,15-pentaazahept- decyl)phenyl)-2,6-dioxo- | 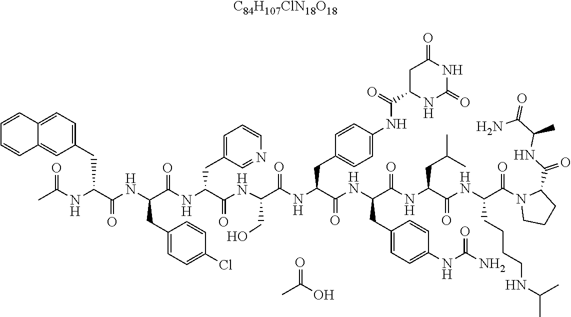 |
| hexahydropyrimidine-4- | |
| carboxamide | |
| Flutamide N-(4-nitro-3-(trifluoro- methyl)phenyl) isobutyramide | 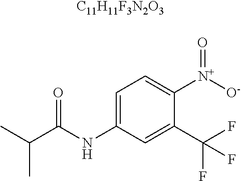 [SMILES: "C11H11F3N2O3" ; CC(C)C(=O)Nc1ccc([N+](=O)[O-])c(C(F)(F)F)c1] |
| Goserelin acetate N-(21-((1H-indol-3-yl) methyl)-1-amino-12-(tert- butoxymethyl)-6-(2-(2- carbamoylhydrazine- carbonyl)pyrrolidine-1- carbonyl)-15-(4-hydroxy- benzyl)-18-(hydroxy- methyl)-25-(1H- imidazol-4-yl)-1-imino-9- isobutyl-8,11,14, 17,20,23-hexaoxo-2,7,10, 13,16,19,22-heptaazapenta- cosan-24-yl)-5-oxo- | 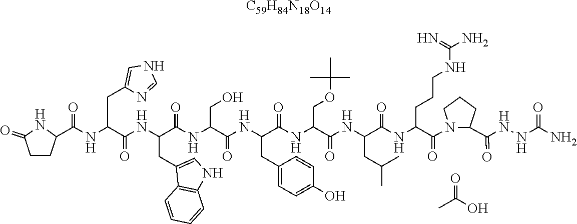 |
| pyrrolidine-2-carboxamide | |
| acetate | |
| Deguelin (7aS,13aS)-13,13a- Dihydro-9,10- dimethoxy-3,3-dimethyl- 3H-bis[1]benzopyrano [3,4-b:6′,5′-e]pyran-7(7aH)- one | 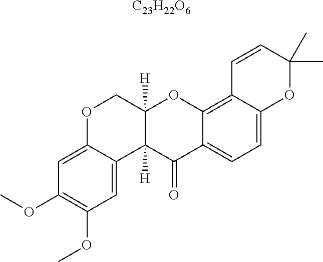 |
| Palomid 529 8-(1-hydroxyethyl)-2- methoxy-3-((4-methoxy- benzyl)oxy)-6H- benzo[c]chromen-6-one | 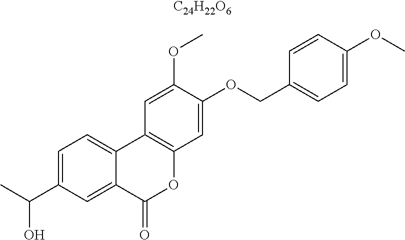 |
| Trametinib N-[3-[3-Cyclopropyl-5- [(2-fluoro-4-iodophenyl) amino]-3,4,6,7- tetrahydro-6,8-dimethyl- 2,4,7-trioxopyrido[4,3- d]pyrimidin-1(2H)-yl] phenyl]acetamide | 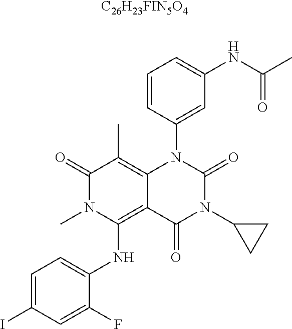 [SMILES: "C26H23FIN5O4" ; CC(=O)Nc1cccc(-n2c(=O)n(C3CC3)c(=O)c3c(Nc4ccc(I)cc4F)n(C)c(=O)c(C)c32)c1] |
| Ceritinib (LDK378) 5-chloro-N2-(2- isopropoxy-5-methyl-4- (piperidin-4-yl)phenyl)-N4- (2-(isopropylsulfonyl) phenyl)pyrimidine-2,4- diamine | 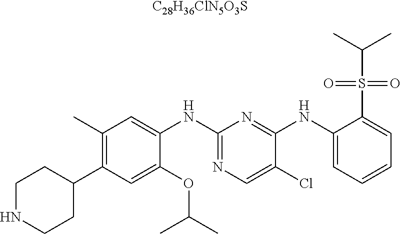 [SMILES: "C28H36ClN5O3S" ; Cc1cc(Nc2ncc(Cl)c(Nc3ccccc3S(=O)(=O)C(C)C)n2)c(OC(C)C)cc1C1CCNCC1] |
| Lanreotide acetate (4S,7S,10S,13R,16S,19S)- 13-((1H-indol-3-yl)methyl)- 19-((R)-2-amino-3- (naphthalen-2-yl) propanamido)-N-((2S,3R)- 1-amino-3-hydroxy-1- oxobutan-2-yl)-10-(4- aminobutyl)-16-(4- hydroxybenzyl)-7- isopropyl-6,9,12,15,18- pentaoxo-1,2-dithia-5,8, 11,14,17-pentaazacyclo- icosane-4-carboxamide acetate | 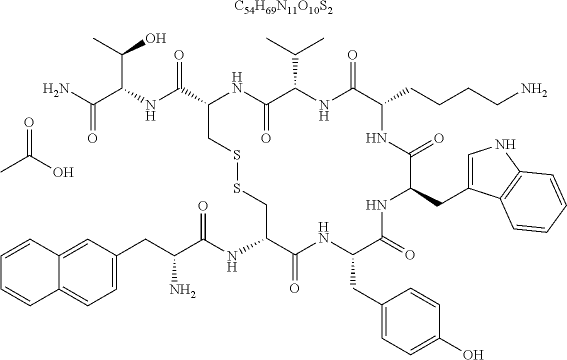 |
| Lenalidomide 3-(4-amino-1- oxoisoindolin-2-yl) piperidine-2,6-dione | 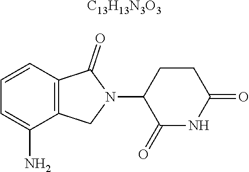 |
| Letrozole 4-[(4-cyanophenyl)-(1,2,4- triazol-1-yl)methyl] benzonitrile | 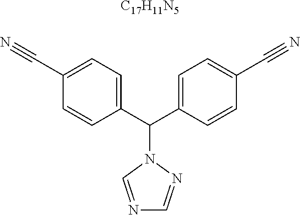 [SMILES: "C17H11N5" ; N#Cc1ccc(C(c2ccc(C#N)cc2)n2cncn2)cc1] |
| Megestrol acetate (8R,9S,10R,13S,14S,17R)- 17-acetyl-6,10,13-trimethyl- 3-oxo-2,3,8,9,10,11,12,13, 14,15,16,17-dodecahydro- 1H-cyclopenta[a] phenanthren-17-yl acetate | 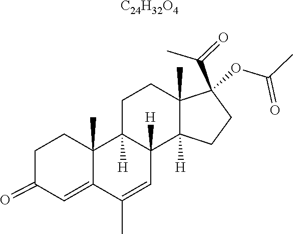 |
| Mesna sodium 2- mercaptoethanesulfonate | 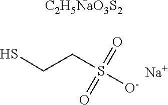 |
| Octreotide (4R,7S,10S,13R,16S,19R)- 13-((1H-indol-3-yl)methyl)- 19-((R)-2-amino-3-phenyl- propanamido)-10-(4- aminobutyl)-16-benzyl- N-((2R,3R)-1,3- dihydroxybutan-2-yl)- 7-(1-hydroxyethyl)- 6,9,12,15,18-pentaoxo- 1,2-dithia-5,8,11,14,17- pentaazacycloicosane-4- carboxamide acetate. | 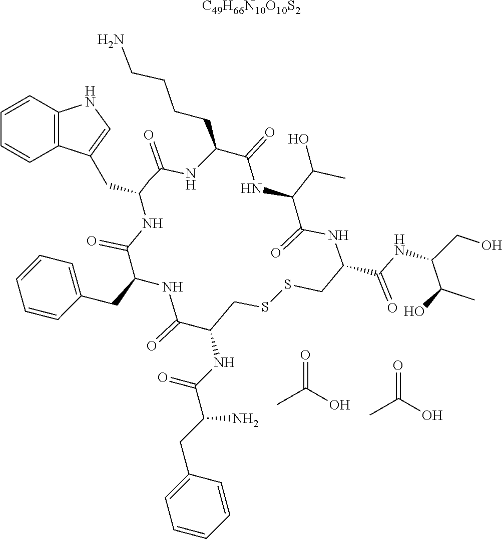 |
| Stilboestrol (4R,7S,10S,13R,16S,19R)- 13-((1H-indol-3-yl)methyl)- 19-((R)-2-amino-3- phenylpropanamido)- 10-(4-aminobutyl)-16- benzyl-N-((2R,3R)-1,3- dihydroxybutan-2-yl)-7-(1- hydroxyethyl)-6,9,12,15,18- pentaoxo-1,2-dithia-5,8,11, 14,17-pentaazacycloico- sane-4-carboxamide acetate. | 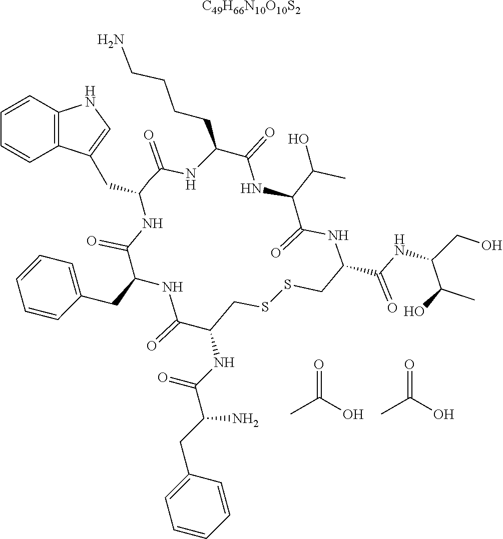 |
| Tamoxifen citrate (Z)-2-(4-(1,2-diphenylbut- 1-en-1-yl)phenoxy)-N,N- dimethylethanamine 2- hydroxypropane-1,2,3- tricarboxylate | 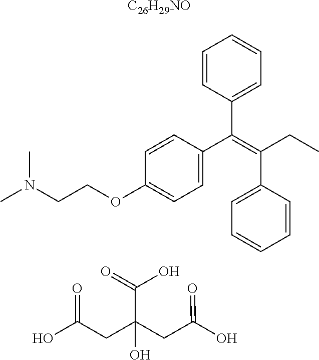 [SMILES: "C26H29NO" ; CC/C(=C(\c1ccccc1)c1ccc(OCCN(C)C)cc1)c1ccccc1 ; O=C(O)CC(O)(CC(=O)O)C(=O)O] |
| Methotrexate (S)-2-(4-(((2,4- diaminopteridin-6- yl)methyl)(methyl)amino) benzamido)pentanedioic acid. | 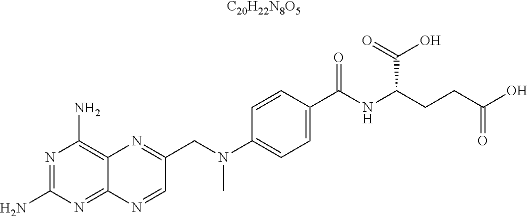 [SMILES: "C20H22N8O5" ; CN(Cc1cnc2nc(N)nc(N)c2n1)c1ccc(C(=O)N[C@@H](CCC(=O)O)C(=O)O)cc1] |
| Leucovorin Calcium 2-(4-(((S)-2-amino-5- formyl-4-oxo-1,4,5,6,7,8- hexahydropteridin-6- yl)(methyl)amino) benzamido)pentanedioic acid | 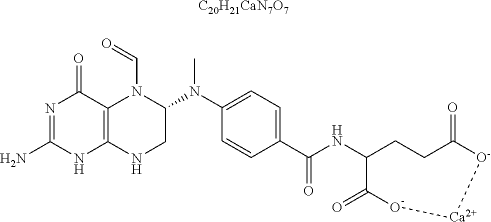 |
| Nolatrexed 2-Amino-6-methyl-5-(4- pyridylsulfanyl) quinazolin-4(3H)-one dihydrochloride | 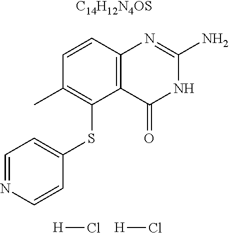 |
| Raltitrexed (S)-2-(5-(methyl((2- methyl-4-oxo-1,4- dihydroquinazolin-6- yl)methyl)amino) thiophene-2- carboxamido) pentanedioic acid | 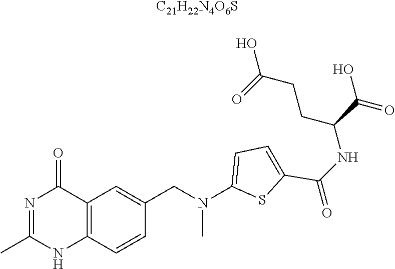 [SMILES: "C21H22N4O6S" ; Cc1nc(=O)c2cc(CN(C)c3ccc(C(=O)N[C@@H](CCC(=O)O)C(=O)O)s3)ccc2[nH]1] |
| Anastrozole 2,2′-(5-((1H-1,2,4- triazol-1-yl)methyl)-1,3- phenylene)bis(2- methylpropanenitrile) | e 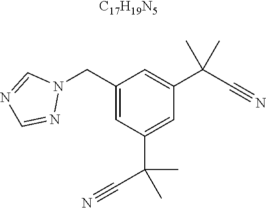 [SMILES: "C17H19N5" ; CC(C)(C#N)c1cc(Cn2cncn2)cc(C(C)(C)C#N)c1] |
| Exemestane (8R,9S,10R,13S,14S)- 10,13-dimethyl-6- methylene-7,8,9,10,11,12, 13,14,15,16-decahydro-3H- cyclopenta[a] phenanthrene-3,17(6H)- dione. | 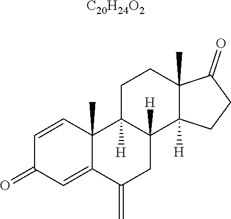 |
| Letrozole 4-[(4-cyanophenyl)-(1,2,4- triazol-1-yl)methyl] benzonitrile | 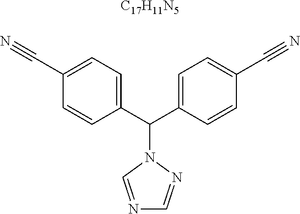 [SMILES: "C17H11N5" ; N#Cc1ccc(C(c2ccc(C#N)cc2)n2cncn2)cc1] |
| KU-55933 2-morpholino-6- (thianthren-1-yl)-4H- pyran-4-one | 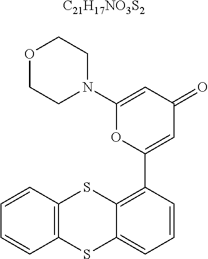 [SMILES: "C21H17NO3S2" ; O=c1cc(-c2cccc3c2Sc2ccccc2S3)oc(N2CCOCC2)c1] |
| Alisertib 4-((9-chloro-7-(2- fluoro-6-methoxyphenyl)- 5H-benzo[c]pyrimido [4,5-e]azepin-2-yl)amino)- 2-methoxybenzoic acid | 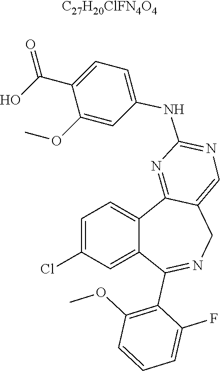 |
| Barasertib (AZD1152) 2-(ethyl(3-(4-(5-(2-(3- fluorophenylamino)-2- oxoethyl)-1H-pyrazol- 3-ylamino)quinazolin-7- yloxy)propyl)amino) ethyl dihydrogen phosphate. | 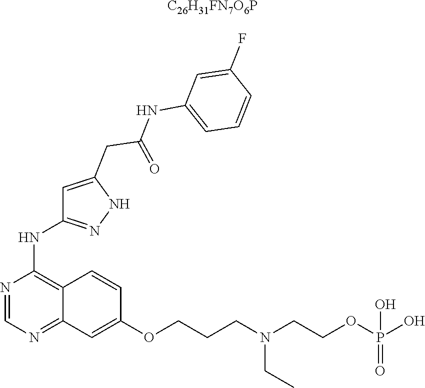 [SMILES: "C26H31FN7O6P" ; CCN(CCCOc1ccc2c(Nc3cc(CC(=O)Nc4cccc(F)c4)[nH]n3)ncnc2c1)CCOP(=O)(O)O] |
| CYC116 4-methyl-5-(2-(4- morpholinophenylamino) pyrimidin-4-yl)thiazol-2- amine | 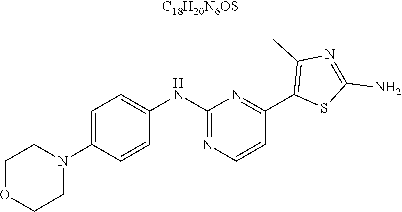 [SMILES: "C18H20N6OS" ; Cc1nc(N)sc1-c1ccnc(Nc2ccc(N3CCOCC3)cc2)n1] |
| INH-13 N-(5-((7-(2-hydroxy-3- (piperidin-1-yl)propoxy)-6- methoxyquinazolin-4-yl) amino)pyrimidin-2-yl) benzamide | 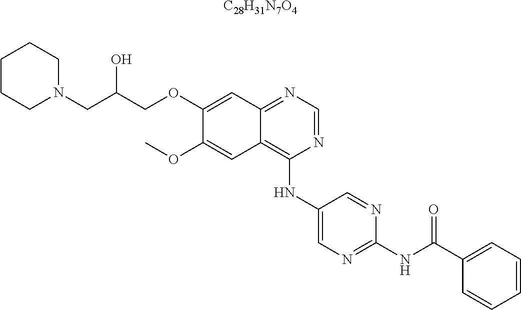 [SMILES: "C28H31N7O4" ; COc1cc2c(Nc3cnc(NC(=O)c4ccccc4)nc3)ncnc2cc1OCC(O)CN1CCCCC1] |
| Hesperadin (Z)-N-(2-oxo-3-(phenyl ((4-(piperidin-1-ylmethyl) phenyl)amino)methylene) indolin-5-yl) ethanesulfonamide | 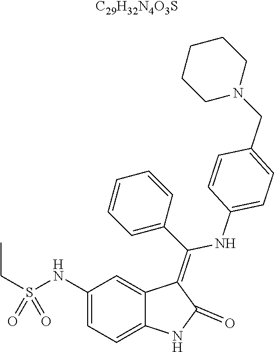 [SMILES: "C29H32N4O3S" ; CCS(=O)(=O)Nc1ccc2c(c1)/C(=C(/Nc1ccc(CN3CCCCC3)cc1)c1ccccc1)C(=O)N2] |
| OM-137 (E)-2-amino-N′-(4- hydroxy-3-methoxy- benzylidene)-4- methylthiazole-5- carbohydrazide | 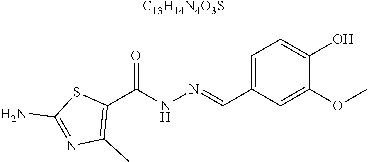 [SMILES: "C13H14N4O3S" ; COc1cc(/C=N/NC(=O)c2sc(N)nc2C)ccc1O] |
| SNS-314 N-(3-Chlorophenyl)-N′-[5- [2-(thieno[3,2-d] pyrimidin-4-ylamino) ethyl]-2-thiazolyl]urea | 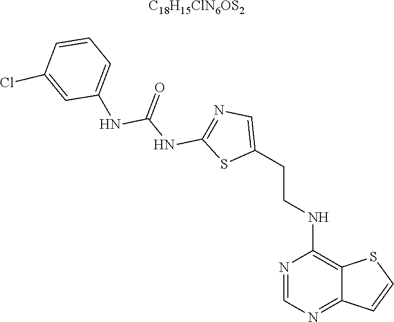 [SMILES: "C18H15ClN6OS2" ; O=C(Nc1cccc(Cl)c1)Nc1ncc(CCNc2ncnc3ccsc23)s1] |
| Obatoclax (Z)-2-(2-((3,5-dimethyl- 1H-pyrrol-2-yl)methylene)- 3-methoxy-2H-pyrrol-5-yl)- 1H-indole methanesulfonate | 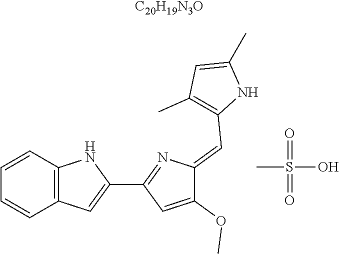 |
| AT-101 1,1′,6,6′,7,7′- hexahydroxy- 5,5′-diisopropyl- 3,3′-dimethyl- [2,2′-binaphthalene]- 8,8′- dicarbaldehyde | 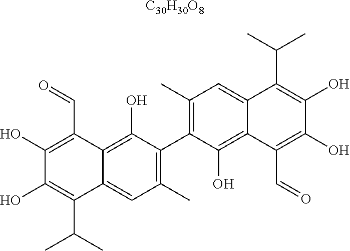 [SMILES: "C30H30O8" ; Cc1cc2c(C(C)C)c(O)c(O)c(C=O)c2c(O)c1-c1c(C)cc2c(C(C)C)c(O)c(O)c(C=O)c2c1O] |
| Aclarubcin Hydrochloride (1R,2R,4S)-methyl 4-(((2R, 5S,6S)-4-(dimethylamino)-5- (((2S,4S,5S,6S)-4- hydroxy-6-methyl-5-(((2R, 6S)-6-methyl-5-oxotetra- hydro-2H-pyran-2-yl)oxy) tetrahydro-2H-pyran-2- yl)oxy)-6-methyl- tetrahydro-2H-pyran-2-yl) oxy)-2-ethyl-2,5,7- trihydroxy-6,11-dioxo- 1,2,3,4,6,11-hexahydro- tetracene-1-carboxylate hydrochloride | 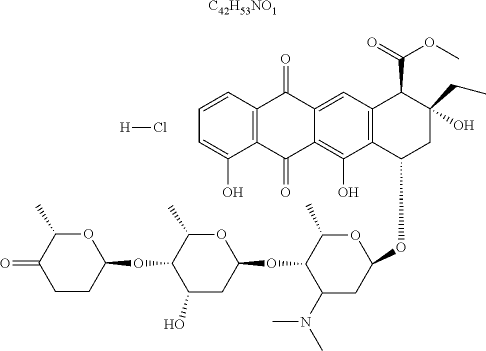 |
| Amrubicin (7S,9S)-9-acetyl-9-amino- 7-(((2S,4S,5R)-4,5- dihydroxytetrahydro-2H- pyran-2-yl)oxy)-6,11- dihydroxy-7,8,9,10- tetrahydrotetracene-5,12- dione hydrochloride | 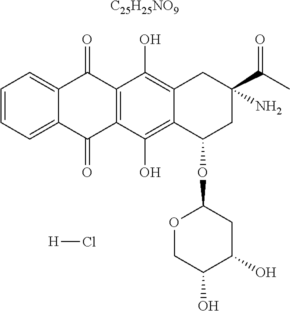 [SMILES: "C25H25NO9" ; CC(=O)[C@]1(N)Cc2c(O)c3c(c(O)c2[C@@H](O[C@H]2C[C@H](O)[C@H](O)CO2)C1)C(=O)c1ccccc1C3=O ; Cl] |
| Annamycin (7S,9S)-7-(((2R,3R,5R,6S)- 4,5-dihydroxy-3-iodo-6- methyltetrahydro-2H- pyran-2-yl)oxy)-6,9,11- trihydroxy-9-(2- hydroxyacetyl)-7,8,9,10- tetrahydrotetracene-5,12- dione | 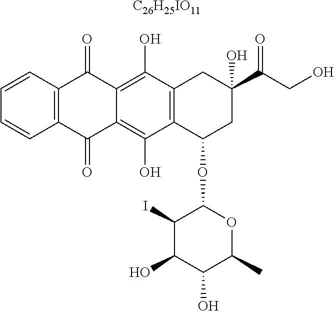 [SMILES: "C26H25IO11" ; C[C@@H]1O[C@@H](O[C@H]2C[C@](O)(C(=O)CO)Cc3c(O)c4c(c(O)c32)C(=O)c2ccccc2C4=O)[C@H](I)[C@H](O)[C@H]1O] |
| Actinomycin D (Dactinomycin) 2-amino-N1,N9-bis(6,13- diisopropyl-2,5,9- trimethyl-1,4,7,11,14- pentaoxohexadecahydro- 1H-pyrrolo[2,1-i][1,4,7,10, 13]oxatetraazacyclohexa- decin-10-yl)-4,6- dimethyl-3-oxo-3H- phenoxazine-1,9- dicarboxamide | 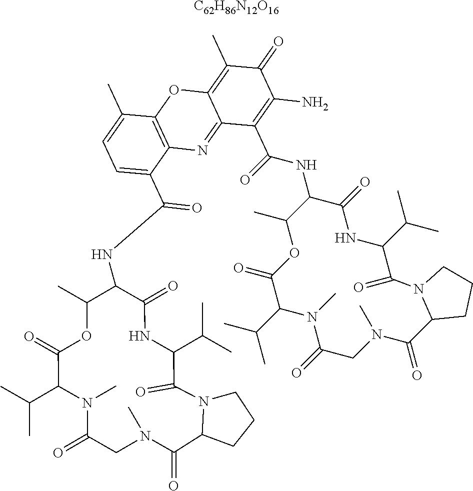 |
| Aldoxorubicin (INNO-206) (E)-N′-(1-((2S,4S)-4- (((2R,5S,6S)-4- amino-5-hydroxy-6- methyltetrahydro-2H-pyran- 2-yl)oxy)-2,5,12-trihydroxy- 7-methoxy-6,11-dioxo- 1,2,3,4,6,11-hexahydro- tetracen-2-yl)-2-hydroxy- ethylidene)-6-(2,5- dioxo-2,5-dihydro-1H- pyrrol-1-yl) hexanehydrazide hydrochloride | 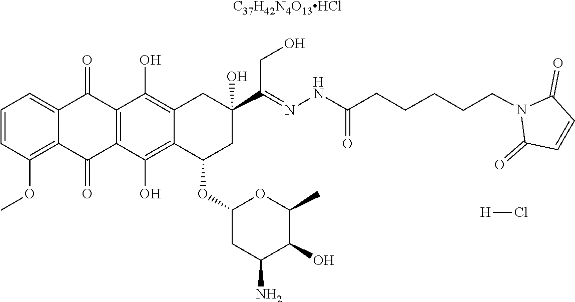 [SMILES: "C37H42N4O13·HCl" ; COc1cccc2c1C(=O)c1c(O)c3c(c(O)c1C2=O)C[C@@](O)(/C(CO)=N/NC(=O)CCCCCN1C(=O)C=CC1=O)C[C@@H]3O[C@H]1C[C@H](N)[C@H](O)[C@H](C)O1 ; Cl] |
| GPX100 (8R,10S)-10-(((2S,4S,5R, 6S)-4-amino-5-hydroxy-6- methyltetrahydro-2H- pyran-2-yl)oxy)-6,8,11- trihydroxy-8-(2- hydroxyethyl)-1-methoxy- 7,8,9,10-tetrahydro- tetracene-5,12- dione | 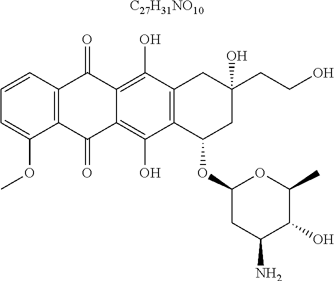 [SMILES: "C27H31NO10" ; COc1cccc2c1C(=O)c1c(O)c3c(c(O)c1C2=O)C[C@@](O)(CCO)C[C@@H]3O[C@@H]1C[C@H](N)[C@@H](O)[C@H](C)O1] |
| Idarubicin hydrochloride (7S,9S)-9-acetyl-7-(((2R, 4S,5S,6S)-4-amino-5- hydroxy-6-methyl- tetrahydro-2H-pyran-2-yl) oxy)-6,9,11-trihydroxy- 7,8,9,10-tetrahydro- tetracene-5,12-dione hydrochloride | 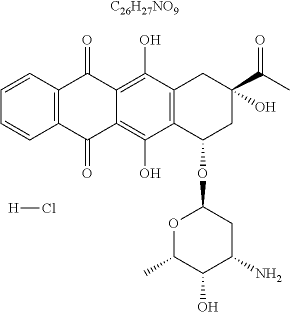 [SMILES: "C26H27NO9" ; CC(=O)[C@]1(O)Cc2c(O)c3c(c(O)c2[C@@H](O[C@H]2C[C@H](N)[C@H](O)[C@H](C)O2)C1)C(=O)c1ccccc1C3=O ; Cl] |
| Ellipticine 5,11-dimethyl-6H-pyrido [4,3-b]carbazole | 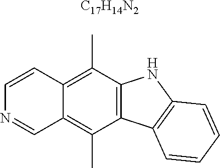 [SMILES: "C17H14N2" ; Cc1c2ccncc2c(C)c2c1[nH]c1ccccc12] |
| Quarfloxin 5-fluoro-N-(2-((S)-1- methylpyrrolidin-2-yl) ethyl)-3-oxo-6- ((R)-3-(pyrazin-2-yl) pyrrolidin-1-yl)-3H- benzo[b]pyrido[3,2,1-kl] phenoxazine-2- carboxamide. | 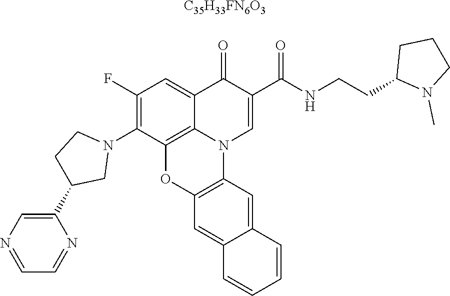 [SMILES: "C35H33FN6O3" ; CN1CCC[C@H]1CCNC(=O)c1cn2c3c(c(N4CC[C@@H](c5cnccn5)C4)c(F)cc3c1=O)Oc1cc3ccccc3cc1-2] |
| EPZ004777 1-(3-((((2R,3S,4R,5R)- 5-(4-amino-7H-pyrrolo [2,3-d]pyimidin-7-yl)- 3,4-dihydroxytetra- hydrofuran-2-yl) methyl)(isopropyl)amino) propyl)-3-(4-(tert- butyl)phenyl)urea | 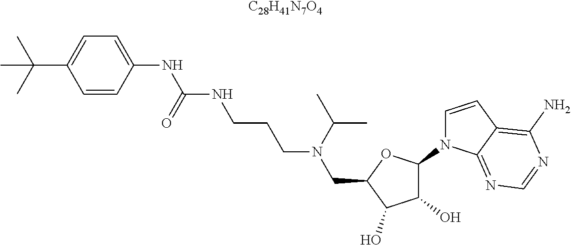 [SMILES: "C28H41N7O4" ; CC(C)N(CCCNC(=O)Nc1ccc(C(C)(C)C)cc1)C[C@H]1O[C@@H](n2ccc3c(N)ncnc32)[C@H](O)[C@@H]1O] |
| Afatinib (S,E)-N-(4-(3-chloro-4- fluorophenylamino)-7- ((tetrahydrofuran-3-yl) methyl)quinazolin-6-yl)- 4-(dimethylamino)but-2- enamide. | 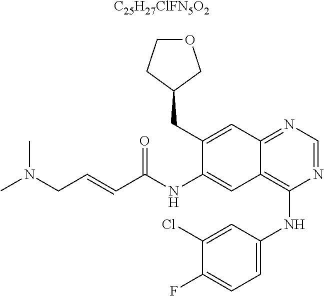 [SMILES: "C25H27ClFN5O2" ; CN(C)C/C=C/C(=O)Nc1cc2c(Nc3ccc(F)c(Cl)c3)ncnc2cc1C[C@H]1CCOC1] |
| Erlotinib hydrochloride N-(3-Ethynylphenyl)-6,7- bis(2-methoxyethoxy)-4- quinazolinamine Monohydrochloride. | 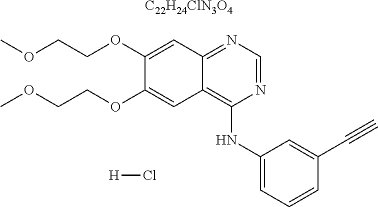 |
| Dacomitinib (E)-N-(4-((3-chloro-4- fluorophenyl)amino)-7- methoxyquinazolin-6-yl)- 4-(piperidin-1-yl)but-2- enamide | 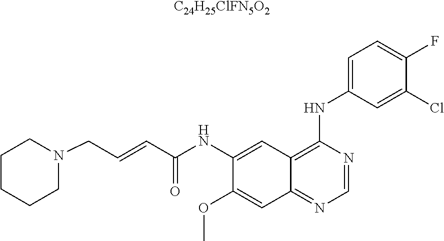 [SMILES: "C24H25ClFN5O2" ; COc1cc2ncnc(Nc3ccc(F)c(Cl)c3)c2cc1NC(=O)/C=C/CN1CCCCC1] |
| TAK-285 N-(2-(4-((3-chloro-4-(3- (trifluoromethyl)phenoxy) phenyl)amino)-5H- pyrrolo[3,2-d] pyrimidin-5-yl)ethyl)-3- hydroxy-3-methyl- butanamid | 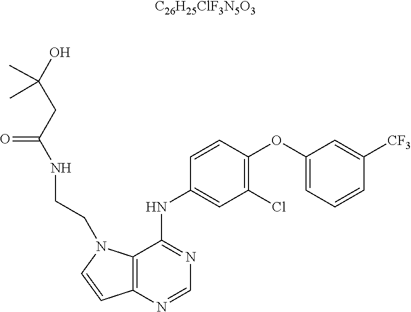 [SMILES: "C26H25ClF3N5O3" ; CC(C)(O)CC(=O)NCCn1ccc2ncnc(Nc3ccc(Oc4cccc(C(F)(F)F)c4)c(Cl)c3)c21] |
| Vandetanib N-(4-bromo-2-fluoro- phenyl)-6-methoxy-7-[(1- methylpiperidin-4-yl) methoxy]quinazolin-4- amine | 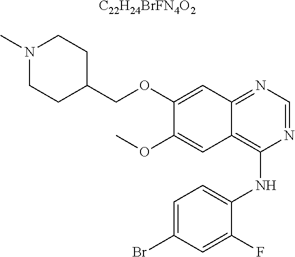 [SMILES: "C22H24BrFN4O2" ; COc1cc2c(Nc3ccc(Br)cc3F)ncnc2cc1OCC1CCN(C)CC1] |
| Defactinib (VS-6063) N-methyl-4-((4-(((3-(N- methylsulfonamido) pyrazin-2-yl)methyl) amino)-5-(trifluoromethyl) pyrimidin-2-yl)amino) benzamide | 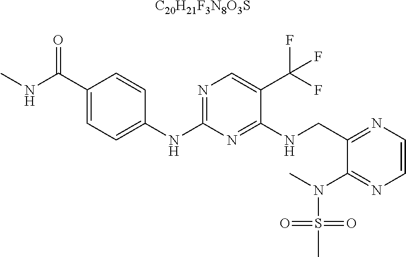 [SMILES: "C20H21F3N8O3S" ; CNC(=O)c1ccc(Nc2ncc(C(F)(F)F)c(NCc3nccnc3N(C)S(C)(=O)=O)n2)cc1] |
| 3-deazaneplanocin A (1S,2R,5R)-5-(4-amino-1H- imidazo[4,5-c]pyridin-1-yl)- 3-(hydroxymethyl) cyclopent-3-ene-1,2- diol hydrochloride | 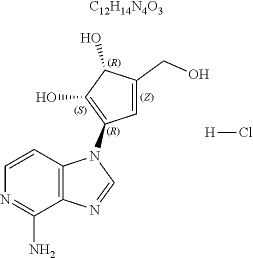 |
| EPZ-005687 1-cyclopent-N-((4,6- dimethyl-2-oxo-1,2- dihydropyridin-3-yl) methyl)-6-(4- (morpholinomethyl) phenyl)-1H-indazole-4- carboxamide. | 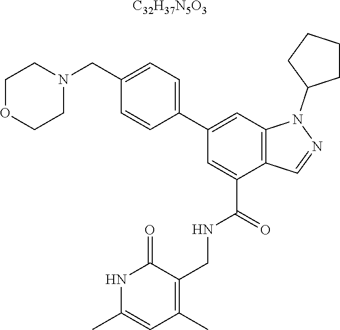 [SMILES: "C32H37N5O3" ; Cc1cc(C)c(CNC(=O)c2cc(-c3ccc(CN4CCOCC4)cc3)cc3c2cnn3C2CCCC2)c(=O)[nH]1] |
| GSK126 (S)-1-(sec-butyl)-N-((4,6- dimethyl-2-oxo-1,2- dihydropyridin-3-yl) methyl)-3-methyl-6-(6- (piperazin-1-yl)pyridin-3- yl)-1H-indole-4- carboxamide | 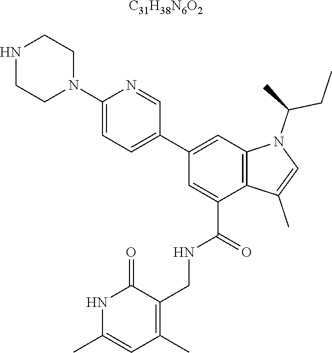 [SMILES: "C31H38N6O2" ; CC[C@H](C)n1cc(C)c2c(C(=O)NCc3c(C)cc(C)[nH]c3=O)cc(-c3ccc(N4CCNCC4)nc3)cc21] |
| UNC-1999 1-isopropyl-6-(6- (4-isopropylpiperazin-1-yl) pyridin-3-yl)-N-((6- methyl-2-oxo-4-propyl-1,2- dihydropyridin-3-yl) methyl)-1H-indazole-4- carboxamide | 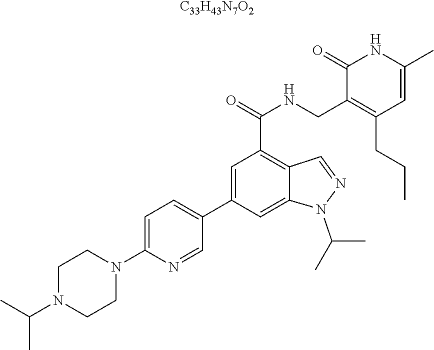 [SMILES: "C33H43N7O2" ; CCCc1cc(C)[nH]c(=O)c1CNC(=O)c1cc(-c2ccc(N3CCN(C(C)C)CC3)nc2)cc2c1cnn2C(C)C] |
| Avagacestat (BMS-708163) (R)-2-(4-chloro-N-(2- fluoro-4-(1,2,4-oxadiazol-3- yl)benzyl)phenyl- sulfonamido)-5,5,5- trifluoropentanamide | 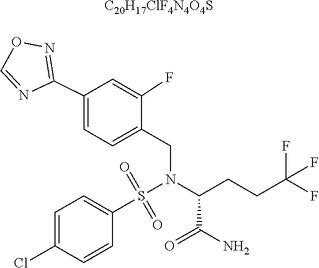 [SMILES: "C20H17ClF4N4O4S" ; NC(=O)[C@@H](CCC(F)(F)F)N(Cc1ccc(-c2ncon2)cc1F)S(=O)(=O)c1ccc(Cl)cc1] |
| BMS-299897 (R)-4-(2-(1-(4-chloro-N- (2,5-difluorophenyl) phenylsulfonamido)ethyl)- 5-fluorophenyl)butanoic acid | 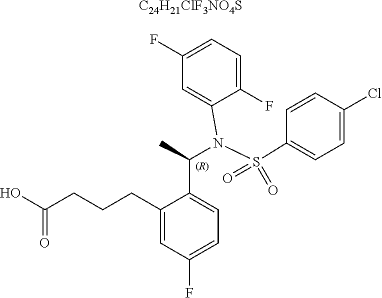 |
| ELND007 (R)-4-cyclopropyl-8- fluoro-5-((6-(trifluoro- methyl)pyridin-3-yl) sulfonyl)-4,5-dihydro-2H- pyrazolo[4,3-c] quinoline | 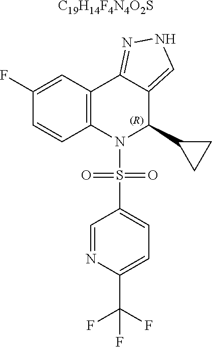 |
| L685458 tert-butyl ((2S,3R,5R)-6- (((S)-1-(((S)-1-amino-1- oxo-3-phenylpropan-2- yl)amino)-4-methyl-1- oxopentan-2-yl)amino)-5- benzyl-3-hydroxy-6-oxo- 1-phenylhexan-2-yl) carbamat | 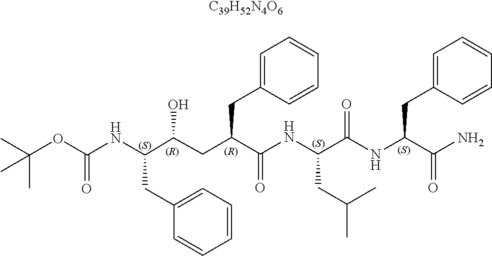 |
| PF-03084014 (S)-2-(((S)-6,8-difluoro- 1,2,3,4-tetrahydro- naphthalen-2-yl)amino)- N-(1-(2-methyl-1- (neopentylamino)propan-2- yl)-1H-imidazol-4-yl) pentanamide | 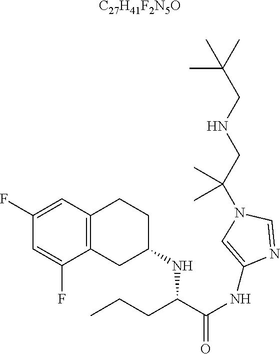 [SMILES: "C27H41F2N5O" ; CCC[C@H](N[C@H]1CCc2cc(F)cc(F)c2C1)C(=O)Nc1cn(C(C)(C)CNCC(C)(C)C)cn1] |
| RO4929097 (S)-2,2-dimethyl-N1-(6- oxo-6,7-dihydro-5H- dibenzo[b,d]azepin-7-yl)- N3-(2,2,3,3,3- pentafluoropropyl) malonamide | 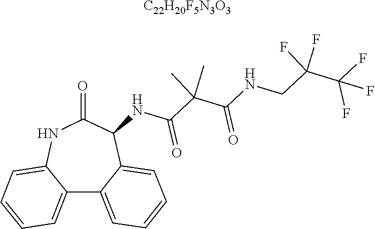 [SMILES: "C22H20F5N3O3" ; CC(C)(C(=O)NCC(F)(F)C(F)(F)F)C(=O)N[C@@H]1C(=O)Nc2ccccc2-c2ccccc21] |
| Semagacestat (S)-2-hydroxy-3-methyl- N-((S)-1-(((S)-3-methyl-2- oxo-2,3,4,5-tetrahydro-1H- benzo[d]azepin-1-yl)amino)- 1-oxopropan-2-yl) butanamide | 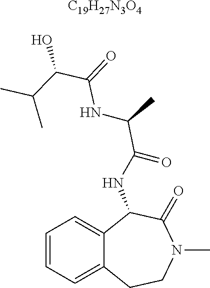 |
| Aroplatin cyclohexane-1,2-diamine; 7,7-dimethyloctanoate; platinum(+2) | 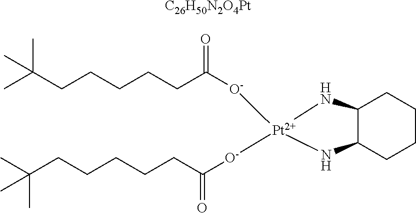 |
| Miriplatin (1R,2R)-cyclohexane-1,2- diamine; platinum(2+); tetradecanoate; hydrate | 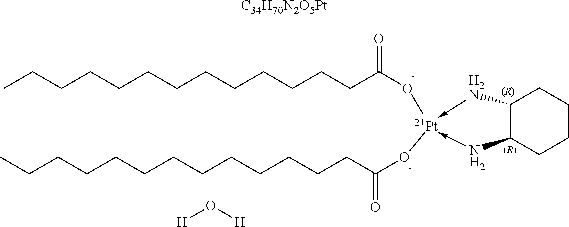 |
| Iproplatin cis, trans, cis-Dichloro- hydroxobis(isopropyl- amine)platinum | 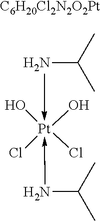 |
| Vinorelbine tartrate (3aR,3a1R,4R,5A,5aR,10bR)- methyl 4-acetoxy-3a-ethyl- 9-((2R,8S)-4-ethyl-8- (methoxycarbonyl)-1,3,6,7, 8,9-hexahydro-2,6- methanoazecino[4,3-b] indol-8-yl)-5-hydroxy-8- methoxy-6-methyl-3a,3a1, 4,5,5a,6,11,12-octahydro- 1H-indolizino[8,1-cd] carbazole-5-carboxylate bis((2R,3R)-2,3- dihydroxysuccinate) | 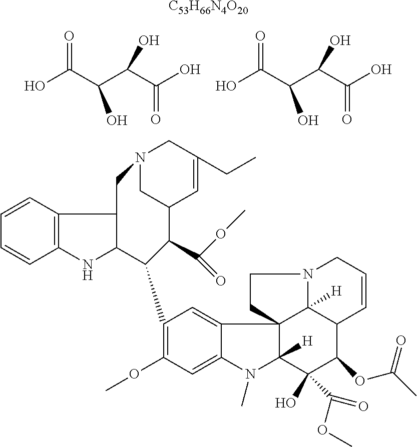 |
| Vindesine (3S,5S,7S,9S)-methyl 9- ((3aR,3a1R,4R,5S,5aR, 10bR)-5-carbamoyl-3a- ethyl-4,5-dihydroxy-8- methoxy-6-methyl-3a,3a1, 4,5,5a,6,11,12-octahydro- 1H-indolizino[8,1-cd] carbazol-9-yl)-5-ethyl-5- hydroxy-2,4,5,6,7,8,9,10- octahydro-1H-3,7- methano[1] azacycloundecino[5,4-b] indole-9-carboxylate | 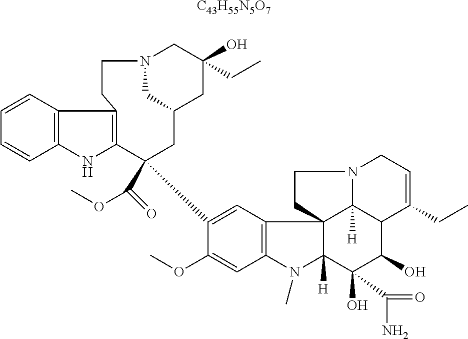 |
| Vinflunine (3aR,3a1R,4R,5S,5aR, 10bR)-methyl 4-acetoxy-9- ((2S,4S,6S,8S)-4-(1,1- difluoroethyl)-8- (methoxycarbonyl)-1,3,4,5, 6,7,8,9-octahydro-2,6- methanoazecino[4,3-b] indol-8-yl)-3a-ethyl-5- hydroxy-8-methoxy-6- methyl-3a,3a1,4,5,5a,6,11, 12-octahydro-1H- indolizino[8,1-cd] carbazole-5-carboxylate | 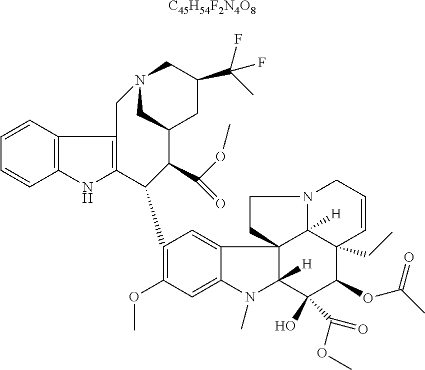 |

Another set of embodiments is generally directed to a device for irreversibly confining a charged compound in an internal region of at least one target cell, including: a fluid containing first target cells and second non-target cells, wherein the first target cells include cytosols having a basic pH property and wherein the second non-target cells include cytosols having a neutral or acidic pH property; and, an uncharged compound adapted to freely pass into the cytosols of the first target cells and the second non-target cells, wherein the uncharged compound is adapted to be converted at a basic pH into at least one charged compound adapted to remain in the cytosols of the first target cells.

In one aspect, the first target cells are cells infected with a virus, such as a coronavirus. In another aspect of the device, the at least one charged compound is adapted to have a detectable quality. In another aspect of the device, the detectable quality is selected from fluorescence, radioactivity or nuclear particle release (e.g., detection of gamma rays, beta particles, and/or alpha particles, etc.), color changes, energy release, electromagnetic radiation release, chemical reactivity, biological reactivity, chemical polarity, solubility changes, etc.

In some cases, the cells may be determined and/or sorted within the device, e.g., as previously discussed. For example, the cells may be determined and/or sorted using a cell cytometer, a fluorescence microscope, a flow cytometer, a cell sorting machine, or the like.

In another aspect, the target cells may be present in a subject, e.g., in vivo. For instance, in one set of embodiments, the target cells are cells infected with a virus. By administering, to a subject, compounds such as those discussed herein, infected cells and non-infected cells may be distinguished in some fashion, e.g., using, for example, radioactivity, color, fluorescence, energy release, chemical reactivity, biological reactivity, chemical polarity, or solubility, e.g., using an appropriate signaling entity. In some cases, systems and methods such as those described herein may be used to determine target cells of interest, and then the target cells of interest (or the non-target cells) may be removed based on the determination. The subject may be, e.g., human or non-human.

Each of the following is incorporated herein by reference in its entirety: U.S. patent application Ser. No. 14/225,512, filed Mar. 26, 2014, entitled “Devices and Methods for Determining and/or Isolating Cells Such as Circulating Cancer or Fetal Cells”; U.S. patent application Ser. No. 14/103,170, filed Dec. 11, 2013, entitled “Devices and Methods for Determining and/or Isolating Cells Such as Circulating Cancer or Fetal Cells”; U.S. patent application Ser. No. 14/312,875, filed Jun. 24, 2014, entitled “Devices and Methods for Determining and/or Isolating Cells Such as Circulating Cancer or Fetal Cells”; and U.S. Pat. Apl. Ser. No. 62/058,884, filed Oct. 2, 2014, entitled “Devices and Methods for Retaining Charged Molecules.” In addition, the following are each incorporated herein by reference in its entirety: U.S. Pat. Nos. 9,709,556, 9,678,076, and 9,063,127, and Int. Pat. Apl. Pub. Nos. WO 2015/200302 and WO 2015/089068.

In addition, U.S. Provisional Patent Application Ser. No. 63/000,435, filed Mar. 26, 2020, entitled “Methods and Systems for Determining Viruses such as Coronaviruses Using pH,” by Bauer, et al., is incorporated herein by reference in its entirety.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. In addition, as used herein the term “about” refers to +/−10%. In addition, when the word “about” is used herein in reference to a number, it should be understood that still another embodiment of the disclosure includes that number not modified by the presence of the word “about.”

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

It is appreciated that certain features, which are, for clarity, described in the context of separate embodiments, may also be provided in combination in a single embodiment. Conversely, various features, which are, for brevity, described in the context of a single embodiment, may also be provided separately or in any suitable subcombination or as suitable in any other described embodiment of the disclosure. Certain features described in the context of various embodiments are not to be considered essential features of those embodiments, unless the embodiment is inoperative without those elements. For example, certain embodiments may include design features that allow for easy attachment and removal of an electrical device to and from a case.

All publications, patents, and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method of determining a coronavirus infection, comprising:
exposing a fluid containing cells arising from a subject to a pH-sensitive entity, wherein the pH-sensitive entity has a first state within cells infected by the coronavirus and a second state within cells not infected by the coronavirus; and
determining the pH-sensitive entity internally within at least some of the cells within the fluid.

2. The method of claim 1, further comprising determining an infection of the subject based on the determination of the pH-sensitive entity internally, and treating the subject if the subject is infected by the coronavirus.

3. The method of claim 1, wherein the cells, when infected by the coronavirus, exhibit a change in pH when compared to the cells not infected by the coronavirus.

4. The method of claim 3, wherein the cells infected by the coronavirus exhibit a change in pH of at least 0.1 pH units when compared to the cells not infected by the coronavirus.

5. The method of claim 1, wherein the coronavirus is COVID-19.

6. The method of claim 1, wherein the fluid comprises blood.

7. The method of claim 1, wherein the fluid comprises saliva.

8. The method of claim 1, wherein the fluid is blood serum, cerebral spinal fluid, urine, nipple aspirate, sputum, pleural or abdominal exudate or transudate.

9. The method of claim 1, further comprising counting the cells in the fluid.

10. The method of claim 1, wherein the pH-sensitive entity comprises one or more of 2′,7′-bis(2-carboxyethyl)-5(6)-carboxyfluorescein tetrakis(acetoxymethyl) ester (BCECF AM), 5(6)-carboxy-2′,7′-dichlorofluorescein, 5(6)-carboxyfluorescein, 5(6)-carboxyfluorescein diacetate, 5(6)-carboxyfluorescein N-hydroxysuccinimide ester, 3,6-diacetoxyphthalonitrile, 6,8-dihydroxy-1,3-pyrenedisulfonic acid disodium salt, Eosin diacetate, or naphthofluorescein.

11. The method of claim 1, wherein the pH-sensitive entity is fluorescent within the cells infected by the coronavirus and is substantially less fluorescent in the cells not infected by the coronavirus.

12. The method of claim 1, wherein the pH-sensitive entity is fluorescent within the cells infected by the coronavirus and is substantially less fluorescent in the cells not infected by the coronavirus.

13. The method of claim 1, further comprising isolating at least some of the cells infected by the coronavirus.

14. The method of claim 13, further comprising performing genetic analysis of the cells infected by the coronavirus.

15. The method of claim 13, comprising isolating at least some of the cells infected by the coronavirus of interest using a flow cytometer.

16. A device, comprising:
a fluid comprising cells arising from a subject;
a pH-sensitive entity internalized within the cells, wherein the pH-sensitive entity has a first state within cells infected by the coronavirus and a second state within cells not infected by the coronavirus; and
a cell cytometer containing the fluid.
